SEQUENCE LISTING (193 sequences)

SEQ ID NO: 1 - protein, 119 aa, synthetic construct
QVQLQQPGAELVKPGASVKLSCKASGYTFTSYWMHWVKQRPGQGLEWIGMIHPNSGTTNYNEKFKNKATLTVDKSSSTAYMQLSSLTSEDSAVYYFTRNGNPRWCFDVWGTGTTVTVSS

SEQ ID NO: 2 - protein, 106 aa, synthetic construct
ENVLTQSPAIMSASPGEKVTVTCSASSSVSYMHWYQQKSSTSPKLWIYDTSKLASGVPGRFSGSGSGNSYSLTISSMEAEDVATYYCFQGSGYPFTFGSGTKLEIK

SEQ ID NO: 3 - protein, 113 aa, synthetic construct
QVQLQQSGAELAKPGASVKMSCKASGYTFTNYWIHWVKQRPGQGLEWIGYISPSTGYTEYNQKFKGKATLTAVKSSSTAFMQLSSLTSEDSAVYFCARGTGYWGQGTTLTVSS

SEQ ID NO: 4 - protein, 107 aa, synthetic construct
DIQMTQSPASLSASVGESVTITCRASENIYSFFAWYQQKEGKSPQLLVYNAKTLEEGVPSRFSGSGSGTQFSLKINSLQPEDFGSYYCQHNYVSPFTFGSGTKLEIK

SEQ ID NO: 5 - protein, 119 aa, synthetic construct
QIQLVQSGPELKKPGETVKISCKASGYTFTNLGLNWVKQAPGKGLKWMGWINTAGGQPTYAEDFKGRCAFSLETSASTAYLQINNLKNEDTATYFCARDDGYYYTMDYWGQGTSVTVSS

SEQ ID NO: 6 - protein, 112 aa, synthetic construct
DIVMTQTPLSLPVSLGDQASISCRSSQSLVHSNGKTYLHWYLQKPGQSPKLLIYKVSNRFSGVPDRFSGSGSGTDFTLKISRVEAEDLGVYFCSQSTHVPWTFGGGTKLEIK

SEQ ID NO: 7 - protein, 118 aa, synthetic construct
QVTLKESGPGILQPSQTLSLTCSFSGFSLSTSGMGVSWIRQPPGKGLEWLAHIYWDDDRRYNPSLKSRLTISRDTSRNQVFLQITTVDTADTATYYCARSPYGSEAYWGQGTLVIVSA

SEQ ID NO: 8 - protein, 107 aa, synthetic construct
MQIVLSQSPTILSAFPGEKVTMTCRASSSVSFIQWYQQKSGSSPKPWIYSPSNLASGVPTRFSGSGSGTSYSLTIGRVEAEDAATYYCQQWSSYPWTFGGGTKVEIK

SEQ ID NO: 9 - protein, 117 aa, synthetic construct
QVQLQQPGAELVKPGASVKMSCKASGYTFTSYWITWVKQRPGQGLEWIGDIYPGSNNTYYNEKFKSKATLTVDTSSSTAYIQLSSLTSEDSAVYYCARLIMSLFDYSGQGTTLTVSS

SEQ ID NO: 10 - protein, 106 aa, synthetic construct
ENVLTQSPANMSASPGEKVTMTCSVSSSVSYIHWYQQKSSTSPTLWIYDTSKLASGVPGRFSGSGSGNSYALTISSMEAEDVATYYCFQGSAYPLTFGAGTKLELK

SEQ ID NO: 11 - protein, 118 aa, synthetic construct
EVQLVESGGDLVKPGGSLKLSCAASGFTFSSYGMSWVRQTPDKRLEWVATISSGGSYIYYPDSMKGRFTISRDNAKNTLYLQMSSLQSEDTAIYYCASDYGSRYFDYWGQGTTLTVSS

SEQ ID NO: 12 - protein, 106 aa, synthetic construct
QIVLTQSPAIMSASPGEKVTMTCSASSSVIYMSWYKQKPGSSPRLLIYDTSNLASGVPVRFSGSGSGTSYSLTISRMEAEDAATYYCQQWSSYPRTFGGGTKLEIK

SEQ ID NO: 13 - protein, 120 aa, synthetic construct
GVQLQQSGAELVKPGASVKLSCTGSGFNIKDPYIHWVKQRPEQGLEWIGRIDPANGNTKYGPRFQGRATMTADTSSNTANLQLSSLTSEDTAVYYCTTWNYYLSRGFDYWGQGTTLTVSS

SEQ ID NO: 14 - protein, 106 aa, synthetic construct
QIVLTQSPAIMSASPGEKVTMTCSARSSVTYMHWYQQKSGTSPKRWIYDTSKLASGVPARFSGSGSGTSYSLTISSMEAEDAATYYCQQWTSNPLTFGAGTKLELK

SEQ ID NO: 15 - protein, 119 aa, synthetic construct
DVQLVESGGGLVQPGGSRKLSCAASGFTFSSFGMHWVRQAPEKGLEWVAYISSGSSTIYYADTVRGRFTISRDNPKNTLFLQMTSLRSEDTAMYYCARGGDYYYALDYWGQGTSVTVSS

SEQ ID NO: 16 - protein, 107 aa, synthetic construct
DIQMTQSPASLSVSVGETVTITCRASENIHSNLEWYQQKQGKSPQLLVYAATNLADGVPSRFSGSGSGTQYSLKINSLQSEDFGSYYCQHFWGTPFTFGSGTKLEIK

SEQ ID NO: 17 - protein, 117 aa, synthetic construct
EVKLVESGGGLVKPGGSLKLSCAASGFTFSSYGMSWVRQTPEKRLEWVATISGGGTYTYYPDSVKGRFTISRDNAKNNLYLQMSSLRSDDTALYYCARHEGLVMDYWGQGTSVTVSS

SEQ ID NO: 18 - protein, 108 aa, synthetic construct
DIQMTQSPASLSASVGETVTITCRASENIYSYLAWYQQKQGKSPQLVVYNAKTLAEGVPSRFSGSGSGTQFSLKINSLQPEDFGTYYCQHHYGPPPYTFGGGTKLEIK

SEQ ID NO: 19 - protein, 117 aa, synthetic construct
QVQLQQPGAELVKPGASVKLSCKASGYTFTSYWMHWVKQRPGQGLEWIGMFHPNSGTTNYNEKFKSKATLTVDKSSSTAYMQLSSLTSEDSAVYYCARRLGIFFDYWGQGTTLTVSS

SEQ ID NO: 20 - protein, 113 aa, synthetic construct
DIVMTQSPSSLTVTAGEKVTMSCRSSQSLLNSGNQKNYLTWYQQKPGQPPKLLIYWASTRDSGVPDRFTGSGSGTDFTLTISSVQTEDLAVYYCQTDYSYPFTFGSGTKLEIK

SEQ ID NO: 21 - protein, 120 aa, synthetic construct
QVQLQQSGPELVRPGVSVKISCKGSGYTFTDYAMHWVKQSHAKSLEWIGIVSTHYGNTNYNQNFKGKATMTVDKSSSTVYMELARLTSEDSAIYYCARSEGLRLDWFAHWGQGTLVTVSA

SEQ ID NO: 22 - protein, 107 aa, synthetic construct
DIQMTQSPASLSASVGEIVTITCRASENIYSYLAWYQQKQGKSPHLLVYNAKTLAEGVPSRFSGSGSGTQFSLKISRLQPEDFGSYYCQNHYVFPFTFGSGTKLEIK

SEQ ID NO: 23 - protein, 119 aa, synthetic construct
QIQLVQSGPELKKPGETVKISCKASGYTFTYYGLNWVKQAPGKGLRWMGWINTSSGVPTYADDFKGRFAISLESSASTVFLEISNLKNEDTAAYFCAKDDGYFYTMDHWGQGTSVTVSS

SEQ ID NO: 24 - protein, 112 aa, synthetic construct
DIVMTQPPLSLPVSLGDQASISCRSSQSLVHNNGKTYLHWYLQKPGQSPKLLIYKVSNRFSGVPDRFSGSGSGTDFTLKISRVETEDLGVYFCSQSTQVPWTFGGGTKLEIK

SEQ ID NO: 25 - protein, 5 aa, synthetic construct
SYWMH

SEQ ID NO: 26 - protein, 17 aa, synthetic construct
MIHPNSGTTNYNEKFKN

SEQ ID NO: 27 - protein, 17 aa, synthetic construct
MIHPQSGTTNYNEKFKN

SEQ ID NO: 28 - protein, 17 aa, synthetic construct
MIHPYSGTTNYNEKFKN

SEQ ID NO: 29 - protein, 10 aa, synthetic construct
NGNPRWCFDV

SEQ ID NO: 30 - protein, 10 aa, synthetic construct
SASSSVSYMH

SEQ ID NO: 31 - protein, 7 aa, synthetic construct
DTSKLAS

SEQ ID NO: 32 - protein, 9 aa, synthetic construct
FQGSGYPFT

SEQ ID NO: 33 - protein, 5 aa, synthetic construct
NYWIH

SEQ ID NO: 34 - protein, 17 aa, synthetic construct
YISPSTGYTEYNQKFKG

SEQ ID NO: 35 - protein, 4 aa, synthetic construct
GTGY

SEQ ID NO: 36 - protein, 11 aa, synthetic construct
RASENIYSFFA

SEQ ID NO: 37 - protein, 7 aa, synthetic construct
NAKTLEE

SEQ ID NO: 38 - protein, 9 aa, synthetic construct
QHNYVSPFT

SEQ ID NO: 39 - protein, 5 aa, synthetic construct
NLGLN

SEQ ID NO: 40 - protein, 17 aa, synthetic construct
WINTAGGQPTYAEDFKG

SEQ ID NO: 41 - protein, 10 aa, synthetic construct
DDGYYYTMDY

SEQ ID NO: 42 - protein, 10 aa, synthetic construct
DDAYYYTMDY

SEQ ID NO: 43 - protein, 10 aa, synthetic construct
DEGYYYTMDY

SEQ ID NO: 44 - protein, 10 aa, synthetic construct
DRGYYYTMDY

SEQ ID NO: 45 - protein, 10 aa, synthetic construct
DLGYYYTMDY

SEQ ID NO: 46 - protein, 10 aa, synthetic construct
DAGYYYTMDY

SEQ ID NO: 47 - protein, 16 aa, synthetic construct
RSSQSLVHSNGKTYLH

SEQ ID NO: 48 - protein, 16 aa, synthetic construct
RSSQSLVHSNAKTYLH

SEQ ID NO: 49 - protein, 16 aa, synthetic construct
RSSQSLVHSQGKTYLH

SEQ ID NO: 50 - protein, 7 aa, synthetic construct
KVSNRFS

SEQ ID NO: 51 - protein, 9 aa, synthetic construct
SQSTHVPWT

SEQ ID NO: 52 - protein, 7 aa, synthetic construct
TSGMGVS

SEQ ID NO: 53 - protein, 16 aa, synthetic construct
HIYWDDDRRYNPSLKS

SEQ ID NO: 54 - protein, 8 aa, synthetic construct
SPYGSEAY

SEQ ID NO: 55 - protein, 10 aa, synthetic construct
RASSSVSFIQ

SEQ ID NO: 56 - protein, 7 aa, synthetic construct
SPSNLAS

SEQ ID NO: 57 - protein, 9 aa, synthetic construct
QQWSSYPWT

SEQ ID NO: 58 - protein, 5 aa, synthetic construct
SYWIT

SEQ ID NO: 59 - protein, 17 aa, synthetic construct
DIYPGSNNTYYNEKFKS

SEQ ID NO: 60 - protein, 8 aa, synthetic construct
LIMSLFDY

SEQ ID NO: 61 - protein, 10 aa, synthetic construct
SVSSSVSYIH

SEQ ID NO: 62 - protein, 7 aa, synthetic construct
DTSKLAS

SEQ ID NO: 63 - protein, 9 aa, synthetic construct
FQGSAYPLT

SEQ ID NO: 64 - protein, 5 aa, synthetic construct
SYGMS

SEQ ID NO: 65 - protein, 17 aa, synthetic construct
TISSGGSYIYYPDSMKG

SEQ ID NO: 66 - protein, 17 aa, synthetic construct
TISSGGSYIYYPDAMKG

SEQ ID NO: 67 - protein, 9 aa, synthetic construct
DYGSRYFDY

SEQ ID NO: 68 - protein, 10 aa, synthetic construct
SASSSVIYMS

SEQ ID NO: 69 - protein, 7 aa, synthetic construct
DTSNLAS

SEQ ID NO: 70 - protein, 9 aa, synthetic construct
QQWSSYPRT

SEQ ID NO: 71 - protein, 5 aa, synthetic construct
DPYIH

SEQ ID NO: 72 - protein, 17 aa, synthetic construct
RIDPANGNTKYGPRFQG

SEQ ID NO: 73 - protein, 17 aa, synthetic construct
RIDPAQGNTKYGPRFQG

SEQ ID NO: 74 - protein, 17 aa, synthetic construct
RIDPANANTKYGPRFQG

SEQ ID NO: 75 - protein, 11 aa, synthetic construct
WNYYLSRGFDY

SEQ ID NO: 76 - protein, 10 aa, synthetic construct
SARSSVTYMH

SEQ ID NO: 77 - protein, 7 aa, synthetic construct
DTSKLAS

SEQ ID NO: 78 - protein, 9 aa, synthetic construct
QQWTSNPLT

SEQ ID NO: 79 - protein, 5 aa, synthetic construct
SFGMH

SEQ ID NO: 80 - protein, 17 aa, synthetic construct
YISSGSSTIYYADTVRG

SEQ ID NO: 81 - protein, 10 aa, synthetic construct
GGDYYYALDY

SEQ ID NO: 82 - protein, 11 aa, synthetic construct
RASENIHSNLE

SEQ ID NO: 83 - protein, 7 aa, synthetic construct
AATNLAD

SEQ ID NO: 84 - protein, 9 aa, synthetic construct
QHFWGTPFT

SEQ ID NO: 85 - protein, 5 aa, synthetic construct
SYGMS

SEQ ID NO: 86 - protein, 17 aa, synthetic construct
TISGGGTYTYYPDSVKG

SEQ ID NO: 87 - protein, 17 aa, synthetic construct
TISGGGTYTYYPDAVKG

SEQ ID NO: 88 - protein, 8 aa, synthetic construct
HEGLVMDY

SEQ ID NO: 89 - protein, 11 aa, synthetic construct
RASENIYSYLA

SEQ ID NO: 90 - protein, 7 aa, synthetic construct
NAKTLAE

SEQ ID NO: 91 - protein, 10 aa, synthetic construct
QHHYGPPPYT

SEQ ID NO: 92 - protein, 5 aa, synthetic construct
SYWMH

SEQ ID NO: 93 - protein, 17 aa, synthetic construct
MFHPNSGTTNYNEKFKS

SEQ ID NO: 94 - protein, 17 aa, synthetic construct
MFHPNAGTTNYNEKFKS

SEQ ID NO: 95 - protein, 17 aa, synthetic construct
MFHPQSGTTNYNEKFKS

SEQ ID NO: 96 - protein, 17 aa, synthetic construct
MFHPYSGTTNYNEKFKS

SEQ ID NO: 97 - protein, 8 aa, synthetic construct
RLGIFFDY

SEQ ID NO: 98 - protein, 17 aa, synthetic construct
RSSQSLLNSGNQKNYLT

SEQ ID NO: 99 - protein, 7 aa, synthetic construct
WASTRDS

SEQ ID NO: 100 - protein, 7 aa, synthetic construct
WASTRDA

SEQ ID NO: 101 - protein, 9 aa, synthetic construct
QTDYSYPFT

SEQ ID NO: 102 - protein, 5 aa, synthetic construct
DYAMH

SEQ ID NO: 103 - protein, 17 aa, synthetic construct
IVSTHYGNTNYNQNFKG

SEQ ID NO: 104 - protein, 11 aa, synthetic construct
SEGLRLDWFAH

SEQ ID NO: 105 - protein, 11 aa, synthetic construct
RASENIYSYLA

SEQ ID NO: 106 - protein, 7 aa, synthetic construct
NAKTLAE

SEQ ID NO: 107 - protein, 9 aa, synthetic construct
QNHYVFPFT

SEQ ID NO: 108 - protein, 5 aa, synthetic construct
YYGLN

SEQ ID NO: 109 - protein, 17 aa, synthetic construct
WINTSSGVPTYADDFKG

SEQ ID NO: 110 - protein, 10 aa, synthetic construct
DDGYFYTMDH

SEQ ID NO: 111 - protein, 10 aa, synthetic construct
DDAYFYTMDH

SEQ ID NO: 112 - protein, 10 aa, synthetic construct
DEGYFYTMDH

SEQ ID NO: 113 - protein, 16 aa, synthetic construct
RSSQSLVHNNGKTYLH

SEQ ID NO: 114 - protein, 16 aa, synthetic construct
RSSQSLVHNNAKTYLH

SEQ ID NO: 115 - protein, 16 aa, synthetic construct
RSSQSLVHNQGKTYLH

SEQ ID NO: 116 - protein, 7 aa, synthetic construct
KVSNRFS

SEQ ID NO: 117 - protein, 9 aa, synthetic construct
SQSTQVPWT

SEQ ID NO: 118 - protein, 122 aa, synthetic construct
EVQLVETGGGVVQPGRSLRLSCAASGFTFSSSGMHWVRQAPGKGLEWVAVISYDVTNKYYVDSVKGRFTISRDNSENTLYLQMNSLRAEDTGVYYCVRDGGPRWLGWYFDLWGRGTLVTVSS

SEQ ID NO: 119 - protein, 107 aa, synthetic construct
NIQMTQSPSSQSASVGDRVTITCRANQAIANSLAWYQQKPGKAPKLLLFGTSLLHSGVPSRFSGSGSGTEYTLTISSLQPDDFGTYYCQQYFSFPRTFGQGTQVDVY

SEQ ID NO: 120 - protein, 122 aa, synthetic construct
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDRYYDILTGTFGYWGQGTLVTVSS

SEQ ID NO: 121 - protein, 106 aa, synthetic construct
EIVMTQSPDFQSVTPKEKVTITCRASQSIGSSLHWYQQKPDQSPKLLIKHASQPISGVPSRFSGSGSGTVFTLTIQSLEAEDAATYYCQQSSRLPTFGGGTKVEIQ

SEQ ID NO: 122 - protein, 122 aa, synthetic construct
QVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHTGYYGSGIGMDVWGQGTLVTVSS

SEQ ID NO: 123 - protein, 110 aa, synthetic construct
HVILTQPPSVSAAPGQKVTISCSGSSSNIGNNYVSWYRQLPGTAPKLLIYDNNQRPSGIPDRFSGSKSGTSATLGITGLQTGDEADYYCATWDSSLGVVVFGGRTKLTVL

SEQ ID NO: 124 - protein, 126 aa, synthetic construct
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSPLQIAVAGYYYGMDVWGQGTTVTVSS

SEQ ID NO: 125 - protein, 110 aa, synthetic construct
NFMLTQPHSVSESPGKTVTISCTRSSGSIVSGYVQWYQQRPGSAPSTVIYEHNQRPSGVPDRFSGSIDSSSNSASLTISGLRTEDEADYYCQSYDSRNMIFGGGTKLTVL

SEQ ID NO: 126 - protein, 5 aa, synthetic construct
SSGMH

SEQ ID NO: 127 - protein, 17 aa, synthetic construct
VISYDVTNKYYVDSVKG

SEQ ID NO: 128 - protein, 17 aa, synthetic construct
VISYDVTNKYYVDAVKG

SEQ ID NO: 129 - protein, 13 aa, synthetic construct
DGGPRWLGWYFDL

SEQ ID NO: 130 - protein, 13 aa, synthetic construct
DAGPRWLGWYFDL

SEQ ID NO: 131 - protein, 13 aa, synthetic construct
EGGPRWLGWYFDL

SEQ ID NO: 132 - protein, 11 aa, synthetic construct
RANQAIANSLA

SEQ ID NO: 133 - protein, 7 aa, synthetic construct
GTSLLHS

SEQ ID NO: 134 - protein, 9 aa, synthetic construct
QQYFSFPRT

SEQ ID NO: 135 - protein, 5 aa, synthetic construct
SYAMS

SEQ ID NO: 136 - protein, 17 aa, synthetic construct
AISGSGGSTYYADSVKG

SEQ ID NO: 137 - protein, 17 aa, synthetic construct
AISGSGGSTYYADAVKG

SEQ ID NO: 138 - protein, 13 aa, synthetic construct
DRYYDILTGTFGY

SEQ ID NO: 139 - protein, 11 aa, synthetic construct
RASQSIGSSLH

SEQ ID NO: 140 - protein, 7 aa, synthetic construct
HASQPIS

SEQ ID NO: 141 - protein, 8 aa, synthetic construct
QQSSRLPT

SEQ ID NO: 142 - protein, 5 aa, synthetic construct
SYWIS

SEQ ID NO: 143 - protein, 17 aa, synthetic construct
RIDPSDSYTNYSPSFQG

SEQ ID NO: 144 - protein, 13 aa, synthetic construct
HTGYYGSGIGMDV

SEQ ID NO: 145 - protein, 13 aa, synthetic construct
SGSSSNIGNNYVS

SEQ ID NO: 146 - protein, 7 aa, synthetic construct
DNNQRPS

SEQ ID NO: 147 - protein, 11 aa, synthetic construct
ATWDSSLGVVV

SEQ ID NO: 148 - protein, 11 aa, synthetic construct
ATWDASLGVVV

SEQ ID NO: 149 - protein, 5 aa, synthetic construct
SYGIS

SEQ ID NO: 150 - protein, 17 aa, synthetic construct
WISAYNGNTNYAQKLQG

SEQ ID NO: 151 - protein, 17 aa, synthetic construct
WISAYQGNTNYAQKLQG

SEQ ID NO: 152 - protein, 17 aa, synthetic construct
WISAYNANTNYAQKLQG

SEQ ID NO: 153 - protein, 17 aa, synthetic construct
DSPLQIAVAGYYYGMDV

SEQ ID NO: 154 - protein, 17 aa, synthetic construct
DAPLQIAVAGYYYGMDV

SEQ ID NO: 155 - protein, 13 aa, synthetic construct
TRSSGSIVSGYVQ

SEQ ID NO: 156 - protein, 7 aa, synthetic construct
EHNQRPS

SEQ ID NO: 157 - protein, 9 aa, synthetic construct
QSYDSRNMI

SEQ ID NO: 158 - protein, 9 aa, synthetic construct
QSYDARNMI

SEQ ID NO: 159 - protein, 119 aa, synthetic construct
QVQLVQSGSELKKPGASVKVSCKASGYTFTNLGLNWVRQAPGQGLEWMGWINTAGGQPTYAEDFKGRFVFSLDTSVSTAYLQISSLKAEDTAVYYCARDDGYYYTMDYWGQGTTVTVSS

SEQ ID NO: 160 - protein, 119 aa, synthetic construct
QVQLVQSGSELKKPGASVKVSCKASGYTFTNLGLNWVRQAPGQGLEWMGWINTAGGQPTYAEDFKGRCVFSLDTSVSTAYLQISSLKAEDTAVYYCARDDGYYYTMDYWGQGTTVTVSS

SEQ ID NO: 161 - protein, 119 aa, synthetic construct
QVQLVQSGSELKKPGASVKVSCKASGYTFTNLGLNWVRQAPGQGLKWMGWINTAGGQPTYAEDFKGRCVFSLDTSVSTAYLQISSLKAEDTAVYFCARDDGYYYTMDYWGQGTTVTVSS

SEQ ID NO: 162 - protein, 119 aa, synthetic construct
QIQLVQSGSELKKPGASVKVSCKASGYTFTNLGLNWVKQAPGQGLKWMGWINTAGGQPTYAEDFKGRCVFSLDTSVSTAYLQISSLKAEDTAVYFCARDDGYYYTMDYWGQGTTVTVSS

SEQ ID NO: 163 - protein, 112 aa, synthetic construct
DIVMTQTPLSLPVTPGEPASISCRSSQSLVHSNGKTYLHWYLQKPGQSPQLLIYKVSNRFSGVPDRFSGSGSGTDFTLKISRVEAEDVGVYYCSQSTHVPWTFGGGTKLEIK

SEQ ID NO: 164 - protein, 112 aa, synthetic construct
DIVMTQTPLSLPVTPGEPASISCRSSQSLVHSNGKTYLHWYLQKPGQSPQLLIYKVSNRFSGVPDRFSGSGSGTDFTLKISRVEAEDVGVYFCSQSTHVPWTFGGGTKLEIK

SEQ ID NO: 165 - protein, 112 aa, synthetic construct
DIVMTQSPLSLPVTPGEPASISCRSSQSLVHSNGKTYLHWYLQKPGQSPQLLIYKVSNRFSGVPDRFSGSGSGTDFTLKISRVEAEDVGVYYCSQSTHVPWTFGGGTKLEIK

SEQ ID NO: 166 - protein, 112 aa, synthetic construct
DIVMTQSPLSLPVTPGEPASISCRSSQSLVHSNGKTYLHWYLQKPGQSPQLLIYKVSNRFSGVPDRFSGSGSGTDFTLKISRVEAEDVGVYFCSQSTHVPWTFGGGTKLEIK

SEQ ID NO: 167 - protein, 118 aa, synthetic construct
QVTLKESGPALVKPTQTLTLTCTFSGFSLSTSGMGVSWIRQPPGKALEWLAHIYWDDDRRYNPSLKSRLTISKDTSKNQVVLTMTNMDPVDTATYYCARSPYGSEAYWGQGTLVTVSS

SEQ ID NO: 168 - protein, 118 aa, synthetic construct
QVTLKESGPALVKPTQTLTLTCTFSGFSLSTSGMGVSWIRQPPGKALEWLAHIYWDDDRRYNPSLKSRLTISRDTSKNQVVLTMTNMDPVDTATYYCARSPYGSEAYWGQGTLVTVSS

SEQ ID NO: 169 - protein, 118 aa, synthetic construct
QVTLKESGPALVKPTQTLTLTCTFSGFSLSTSGMGVSWIRQPPGKGLEWLAHIYWDDDRRYNPSLKSRLTISRDTSKNQVVLTITNMDPVDTATYYCARSPYGSEAYWGQGTLVTVSS

SEQ ID NO: 170 - protein, 106 aa, synthetic construct
EIVLTQSPDFQSVTPKEKVTITCRASSSVSFIQWYQQKPDQSPKLWIKSPSNLASGVPSRFSGSGSGTDFTLTINSLEAEDAATYYCQQWSSYPWTFGQGTKVEIK

SEQ ID NO: 171 - protein, 106 aa, synthetic construct
EIVLTQSPDFQSVTPKEKVTITCRASSSVSFIQWYQQKPDQSPKPWIKSPSNLASGVPSRFSGSGSGTDFTLTINSLEAEDAATYYCQQWSSYPWTFGQGTKVEIK

SEQ ID NO: 172 - protein, 106 aa, synthetic construct
EIVLTQSPDFQSVTPKEKVTITCRASSSVSFIQWYQQKPDQSPKPWIYSPSNLASGVPSRFSGSGSGTDFTLTINSLEAEDAATYYCQQWSSYPWTFGQGTKVEIK

SEQ ID NO: 173 - protein, 106 aa, synthetic construct
EIVLSQSPDFQSVTPKEKVTITCRASSSVSFIQWYQQKPDQSPKPWIYSPSNLASGVPSRFSGSGSGTDYTLTINSLEAEDAATYYCQQWSSYPWTFGQGTKVEIK

SEQ ID NO: 174 - protein, 119 aa, synthetic construct
QVQLVQSGSELKKPGASVKVSCKASGYTFTNLGLNWVRQAPGQGLEWMGWINTAGGQPTYAEDFKGRFVFSLDTSVSTAYLQISSLKAEDTAVYYCARDEGYYYTMDYWGQGTTVTVSS

SEQ ID NO: 175 - protein, 119 aa, synthetic construct
QVQLVQSGSELKKPGASVKVSCKASGYTFTNLGLNWVRQAPGQGLEWMGWINTAGGQPTYAEDFKGRFVFSLDTSVSTAYLQISSLKAEDTAVYYCARDRGYYYTMDYWGQGTTVTVSS

SEQ ID NO: 176 - protein, 119 aa, synthetic construct
QVQLVQSGSELKKPGASVKVSCKASGYTFTNLGLNWVRQAPGQGLEWMGWINTAGGQPTYAEDFKGRFVFSLDTSVSTAYLQISSLKAEDTAVYYCARDLGYYYTMDYWGQGTTVTVSS

SEQ ID NO: 177 - protein, 119 aa, synthetic construct
QVQLVQSGSELKKPGASVKVSCKASGYTFTNLGLNWVRQAPGQGLEWMGWINTAGGQPTYAEDFKGRFVFSLDTSVSTAYLQISSLKAEDTAVYYCARDAGYYYTMDYWGQGTTVTVSS

SEQ ID NO: 178 - protein, 112 aa, synthetic construct
DIVMTQSPLSLPVTPGEPASISCRSSQSLVHSNAKTYLHWYLQKPGQSPQLLIYKVSNRFSGVPDRFSGSGSGTDFTLKISRVEAEDVGVYYCSQSTHVPWTFGGGTKLEIK

SEQ ID NO: 179 - protein, 112 aa, synthetic construct
DIVMTQSPLSLPVTPGEPASISCRSSQSLVHSQGKTYLHWYLQKPGQSPQLLIYKVSNRFSGVPDRFSGSGSGTDFTLKISRVEAEDVGVYYCSQSTHVPWTFGGGTKLEIK

SEQ ID NO: 180 - protein, 117 aa, synthetic construct
QVQLQQPGAELVKPGASVKLSCKASGYTFTSYWMHWVNQRPGQGLEWIGMIHPNSGSTNYNEKFKSKATLTVDKSSSTAYMQLSSLTSEDSAVYYCARNYGRYFDYWGQGTTLTVSS

SEQ ID NO: 181 - protein, 130 aa, synthetic construct
MDFQVQIFSFLLISVSVIMSRGQIVLTQSPAIMSASPGEKVTLTCSANSSVSSSSLYWYQQKPGSSPKLWIYSTSNLASGVPARFSGSGSGTSYSLTISSMEAEDAASYFCHQWSSYPLTFGAGTKLELK

SEQ ID NO: 182 - protein, 5 aa, synthetic construct
SYWMH

SEQ ID NO: 183 - protein, 17 aa, synthetic construct
MIHPNSGSTNYNEKFKS

SEQ ID NO: 184 - protein, 17 aa, synthetic construct
MIHPNAGSTNYNEKFKS

SEQ ID NO: 185 - protein, 17 aa, synthetic construct
MIHPQSGSTNYNEKFKS

SEQ ID NO: 186 - protein, 17 aa, synthetic construct
MIHPYSGSTNYNEKFKS

SEQ ID NO: 187 - protein, 8 aa, synthetic construct
NYGRYFDY

SEQ ID NO: 188 - protein, 12 aa, synthetic construct
SANSSVSSSSLY

SEQ ID NO: 189 - protein, 12 aa, synthetic construct
SANASVSSSSLY

SEQ ID NO: 190 - protein, 12 aa, synthetic construct
SAQSSVSSSSLY

SEQ ID NO: 191 - protein, 12 aa, synthetic construct
SAYSSVSSSSLY

SEQ ID NO: 192 - protein, 7 aa, synthetic construct
STSNLAS

SEQ ID NO: 193 - protein, 9 aa, synthetic construct
HQWSSYPLT

(19) United States
(12) Patent Application Publication
(10) Pub. No.: US 2025/0313632 A1
(43) Pub. Date: Oct. 9, 2025

(54) ANTI-B7-H3 ANTIBODIES AND USES THEREOF

(71) Applicants: CONCEPT TO MEDICINE BIOTECH CO., LTD., Shanghai (CN); LEPU BIOPHARMA CO., LTD., Shanghai (CN)

(72) Inventors: Peng ZENG, Shanghai (CN); Zhihu QU, Shanghai (CN); Wenqing JIANG, Shanghai (CN)

(21) Appl. No.: 18/860,545

(22) PCT Filed: Apr. 26, 2023
(86) PCT No.: PCT/CN2023/090822

§ 371 Date: Oct. 25, 2024

(30) Foreign Application Priority Data
Apr. 26, 2022 (WO) PCT/CN2022/089391

(51) Int. Cl. C07K 16/28; A61K 47/68

(52) U.S. Cl. CPC C07K 16/2827; A61K 47/68031; A61K 47/6851; C07K 2317/24; C07K 2317/40; C07K 2317/77; C07K 2317/92

(57) ABSTRACT

This disclosure provides anti-B7-H3 antibodies, including murine antibodies, humanized antibodies, and human antibodies, as well as those with further optimized CDR sequences. These antibodies are suitable for use in treating diseases such as cancer.

### BACKGROUND

The B7 is a family of integral membrane proteins found on activated antigen-presenting cells (APC). When paired with either a CD28 or CD152 (CTLA-4) surface protein on a T cell, a B7 protein can produce a costimulatory signal or a coinhibitory signal to enhance or decrease the activity of an MHC-TCR signal between the APC and the T cell, respectively. The binding of the B7 of APC to CTLA-4 of T-cells causes inhibition of the activity of T-cells.

There are two major types of B7 proteins: B7-1 or CD80, and B7-2 or CD86. The proteins CD28 and CTLA-4 (CD152) each interact with both B7-1 and B7-2. Other proteins in this family include B7-DC (PD-L2), B7-H1 (PD-L1), B7-H2 (ICOSLG), B7-H3 (CD276), B7-H4 (VTCN1), B7-H5 (VISTA), B7-H6, and B7-H7.

B7-H3 is a type I transmembrane protein, which contains extracellular immunoglobulin-like variable regions (IgV) and constant regions (IgC), a transmembrane region, and a short cytoplasmic region. B7-H3 has two splicing variants, B7-H3a and B7-H3b. The extracellular domain of B7-H3a includes two immunoglobulin domains of IgV-IgC (also known as 2IgB7-H3), while the extracellular domain of B7-H3b consists of four immunoglobulin domains of IgV-IgC-IgV-IgC (also known as 4IgB7-H3). 4IgB7-H3 is the predominant isoform in humans while 2IgB7-H3 is the only isoform in mice.

B7-H3 protein is not expressed or is poorly expressed in normal tissues and cells, but highly expressed in various tumor tissues and is closely correlated with tumor progression, patient survival and prognosis. It has been clinically reported that B7-H3 is over-expressed in many types of cancers, especially in non-small cell lung cancer, renal cancer, urinary tract epithelial cancer, colorectal cancer, prostate cancer, glioblastoma multiforme, ovarian cancer and pancreas cancer. In addition, it has also been reported in the literature that, in prostate cancer, the expression level of B7-H3 is positively correlated with clinical pathological malignancy and cancer progression. Likewise, in glioblastoma multiforme, B7-H3 expression is inversely associated with event-free survival, and in pancreatic cancer, the expression of B7-H3 is associated with lymph node metastasis and pathological progression. Therefore, B7-H3 is a suitable potential therapeutic target. It has been demonstrated that antibodies targeting B7-H3 can enhance infiltrative CD8-positive T cells in tumors and inhibit tumor growth.

### SUMMARY

The present disclosure, in various embodiments, provides antibodies and antigen-binding fragments specific to the human B7-H3 protein. Both murine antibodies with their humanized counterparts and full human antibodies have been identified. These antibodies generally have superior properties to the benchmark antibody MGA017 (MacroGenics). These antibodies, along with molecules derived therefrom such as chimeric antigen receptors (CAR), multi-specific antibodies, can be suitably used for treating diseases such as cancer.

Accordingly, one embodiment of the present disclosure provides an antibody or antigen-binding fragment thereof which has specificity to the human B7-H3 (CD276) protein and comprises a heavy chain variable region (VH) comprising a VH CDR1, a VH CDR2 and a VH CDR3, and a light chain variable region (VL) comprising a VL CDR1, a VL CDR2, and a VL CDR3.

In some embodiments, the VH CDR1 comprises the amino acid sequence of SEQ ID NO: 25; the VH CDR2 comprises the amino acid sequence of SEQ ID NO: 26, 27, or 28; the VH CDR3 comprises the amino acid sequence of SEQ ID NO: 29; the VL CDR1 comprises the amino acid sequence of SEQ ID NO: 30; the VL CDR2 comprises the amino acid sequence of SEQ ID NO: 31; and the VL CDR3 comprises the amino acid sequence of SEQ ID NO: 32.

In some embodiments, the VH CDR1 comprises the amino acid sequence of SEQ ID NO: 33; the VH CDR2 comprises the amino acid sequence of SEQ ID NO: 34; the VH CDR3 comprises the amino acid sequence of SEQ ID NO: 35; the VL CDR1 comprises the amino acid sequence of SEQ ID NO: 36; the VL CDR2 comprises the amino acid sequence of SEQ ID NO: 37; and the VL CDR3 comprises the amino acid sequence of SEQ ID NO: 38.

In some embodiments, the VH CDR1 comprises the amino acid sequence of SEQ ID NO: 39; the VH CDR2 comprises the amino acid sequence of SEQ ID NO: 40; the VH CDR3 comprises the amino acid sequence of SEQ ID NO: 41, 42, 43, 44, 45 or 46; the VL CDR1 comprises the amino acid sequence of SEQ ID NO: 47, 48 or 49; the VL CDR2 comprises the amino acid sequence of SEQ ID NO: 50; and the VL CDR3 comprises the amino acid sequence of SEQ ID NO: 51.

In some embodiments, the VH CDR1 comprises the amino acid sequence of SEQ ID NO: 52; the VH CDR2 comprises the amino acid sequence of SEQ ID NO: 53; the VH CDR3 comprises the amino acid sequence of SEQ ID NO: 54; the VL CDR1 comprises the amino acid sequence of SEQ ID NO: 55; the VL CDR2 comprises the amino acid sequence of SEQ ID NO: 56; and the VL CDR3 comprises the amino acid sequence of SEQ ID NO: 57.

In some embodiments, the VH CDR1 comprises the amino acid sequence of SEQ ID NO: 58; the VH CDR2 comprises the amino acid sequence of SEQ ID NO: 59; the VH CDR3 comprises the amino acid sequence of SEQ ID NO: 60; the VL CDR1 comprises the amino acid sequence of SEQ ID NO: 61; the VL CDR2 comprises the amino acid sequence of SEQ ID NO: 62; and the VL CDR3 comprises the amino acid sequence of SEQ ID NO: 63.

In some embodiments, the VH CDR1 comprises the amino acid sequence of SEQ ID NO: 64; the VH CDR2 comprises the amino acid sequence of SEQ ID NO: 65 or 66; the VH CDR3 comprises the amino acid sequence of SEQ ID NO: 67; the VL CDR1 comprises the amino acid sequence of SEQ ID NO: 68; the VL CDR2 comprises the amino acid sequence of SEQ ID NO: 69; and the VL CDR3 comprises the amino acid sequence of SEQ ID NO: 70.

In some embodiments, the VH CDR1 comprises the amino acid sequence of SEQ ID NO: 71; the VH CDR2 comprises the amino acid sequence of SEQ ID NO: 72, 73 or 74; the VH CDR3 comprises the amino acid sequence of SEQ ID NO: 75; the VL CDR1 comprises the amino acid sequence of SEQ ID NO: 76; the VL CDR2 comprises the amino acid sequence of SEQ ID NO: 77; and the VL CDR3 comprises the amino acid sequence of SEQ ID NO: 78.

In some embodiments, the VH CDR1 comprises the amino acid sequence of SEQ ID NO: 79; the VH CDR2 comprises the amino acid sequence of SEQ ID NO: 80; the VH CDR3 comprises the amino acid sequence of SEQ ID NO: 81; the VL CDR1 comprises the amino acid sequence of SEQ ID NO: 82; the VL CDR2 comprises the amino acid sequence of SEQ ID NO: 83; and the VL CDR3 comprises the amino acid sequence of SEQ ID NO: 84.

In some embodiments, the VH CDR1 comprises the amino acid sequence of SEQ ID NO: 85; the VH CDR2 comprises the amino acid sequence of SEQ ID NO: 86 or 87; the VH CDR3 comprises the amino acid sequence of SEQ ID NO: 88; the VL CDR1 comprises the amino acid sequence of SEQ ID NO: 89; the VL CDR2 comprises the amino acid sequence of SEQ ID NO: 90; and the VL CDR3 comprises the amino acid sequence of SEQ ID NO: 91.

In some embodiments, the VH CDR1 comprises the amino acid sequence of SEQ ID NO: 92; the VH CDR2 comprises the amino acid sequence of SEQ ID NO: 93, 94, 95 or 96; the VH CDR3 comprises the amino acid sequence of SEQ ID NO: 97; the VL CDR1 comprises the amino acid sequence of SEQ ID NO: 98; the VL CDR2 comprises the amino acid sequence of SEQ ID NO: 99 or 100; and the VL CDR3 comprises the amino acid sequence of SEQ ID NO: 101.

In some embodiments, the VH CDR1 comprises the amino acid sequence of SEQ ID NO: 102; the VH CDR2 comprises the amino acid sequence of SEQ ID NO: 103; the VH CDR3 comprises the amino acid sequence of SEQ ID NO: 104; the VL CDR1 comprises the amino acid sequence of SEQ ID NO: 105; the VL CDR2 comprises the amino acid sequence of SEQ ID NO: 106; and the VL CDR3 comprises the amino acid sequence of SEQ ID NO: 107.

In some embodiments, the VH CDR1 comprises the amino acid sequence of SEQ ID NO: 108; the VH CDR2 comprises the amino acid sequence of SEQ ID NO: 109; the VH CDR3 comprises the amino acid sequence of SEQ ID NO: 110, 111 or 112; the VL CDR1 comprises the amino acid sequence of SEQ ID NO: 113, 114 or 115; the VL CDR2 comprises the amino acid sequence of SEQ ID NO: 116; and the VL CDR3 comprises the amino acid sequence of SEQ ID NO: 117.

In some embodiments, the VH CDR1 comprises the amino acid sequence of SEQ ID NO: 182; the VH CDR2 comprises the amino acid sequence of SEQ ID NO: 183, 184, 185, or 186; the VH CDR3 comprises the amino acid sequence of SEQ ID NO: 187; the VL CDR1 comprises the amino acid sequence of SEQ ID NO: 188, 189, 190, or 191; the VL CDR2 comprises the amino acid sequence of SEQ ID NO: 192; and the VL CDR3 comprises the amino acid sequence of SEQ ID NO: 193.

In some embodiments, the VH CDR1 comprises the amino acid sequence of SEQ ID NO: 39; the VH CDR2 comprises the amino acid sequence of SEQ ID NO: 40; the VH CDR3 comprises the amino acid sequence of SEQ ID NO: 41; the VL CDR1 comprises the amino acid sequence of SEQ ID NO: 47; the VL CDR2 comprises the amino acid sequence of SEQ ID NO: 50; and the VL CDR3 comprises the amino acid sequence of SEQ ID NO: 51.

In some embodiments, the VH comprises an amino acid sequence selected from the group consisting of SEQ ID NO: 5 and 159-162 and the VL comprises an amino acid sequence selected from the group consisting of SEQ ID NO: 6 and 163-166. In some embodiments, the VH comprises the amino acid sequence of SEQ ID NO: 159 and the VL comprises the amino acid sequence of SEQ ID NO: 165.

In some embodiments, the VH CDR1 comprises the amino acid sequence of SEQ ID NO: 39; the VH CDR2 comprises the amino acid sequence of SEQ ID NO: 40; the VH CDR3 comprises the amino acid sequence of SEQ ID NO: 41; the VL CDR1 comprises the amino acid sequence of SEQ ID NO: 48 or 49; the VL CDR2 comprises the amino acid sequence of SEQ ID NO: 50; and the VL CDR3 comprises the amino acid sequence of SEQ ID NO: 51.

In some embodiments, the VH comprises the amino acid of SEQ ID NO: 159 and the VL comprises the amino acid sequence of SEQ ID NO: 178. In some embodiments, the VH comprises the amino acid of SEQ ID NO: 159 and the VL comprises the amino acid sequence of SEQ ID NO: 179.

In some embodiments, the VH CDR1 comprises the amino acid sequence of SEQ ID NO: 52; the VH CDR2 comprises the amino acid sequence of SEQ ID NO: 53; the VH CDR3 comprises the amino acid sequence of SEQ ID NO: 54; the VL CDR1 comprises the amino acid sequence of SEQ ID NO: 55; the VL CDR2 comprises the amino acid sequence of SEQ ID NO: 56; and the VL CDR3 comprises the amino acid sequence of SEQ ID NO: 57.

In some embodiments, the VH comprises an amino acid sequence selected from the group consisting of SEQ ID NO: 7 and 167-169 and the VL comprises an amino acid sequence selected from the group consisting of SEQ ID NO: 8 and 170-173.

In some embodiments, the VH CDR1 comprises the amino acid sequence of SEQ ID NO: 33; the VH CDR2 comprises the amino acid sequence of SEQ ID NO: 34; the VH CDR3 comprises the amino acid sequence of SEQ ID NO: 35; the VL CDR1 comprises the amino acid sequence of SEQ ID NO: 36; the VL CDR2 comprises the amino acid sequence of SEQ ID NO: 37; and the VL CDR3 comprises the amino acid sequence of SEQ ID NO: 38.

In some embodiments, the VH comprises the amino acid sequence of SEQ ID NO: 3 and the VL comprises the amino acid sequence of SEQ ID NO: 4.

Also provided, in one embodiment, is an antibody or antigen-binding fragment thereof which has specificity to the human B7-H3 (CD276) protein and comprises a heavy chain variable region (VH) comprising a VH CDR1, a VH CDR2 and a VH CDR3, and a light chain variable region (VL) comprising a VL CDR1, a VL CDR2, and a VL CDR3.

In some embodiments, the VH CDR1 comprises the amino acid sequence of SEQ ID NO: 126; the VH CDR2 comprises the amino acid sequence of SEQ ID NO: 127 or 128; the VH CDR3 comprises the amino acid sequence of SEQ ID NO: 129, 130 or 131; the VL CDR1 comprises the amino acid sequence of SEQ ID NO: 132; the VL CDR2 comprises the amino acid sequence of SEQ ID NO: 133; and the VL CDR3 comprises the amino acid sequence of SEQ ID NO: 134.

In some embodiments, the VH CDR1 comprises the amino acid sequence of SEQ ID NO: 135; the VH CDR2 comprises the amino acid sequence of SEQ ID NO: 136 or 137; the VH CDR3 comprises the amino acid sequence of SEQ ID NO: 138; the VL CDR1 comprises the amino acid sequence of SEQ ID NO: 139; the VL CDR2 comprises the amino acid sequence of SEQ ID NO: 140; and the VL CDR3 comprises the amino acid sequence of SEQ ID NO: 141

In some embodiments, the VH CDR1 comprises the amino acid sequence of SEQ ID NO: 142; the VH CDR2 comprises the amino acid sequence of SEQ ID NO: 143; the VH CDR3 comprises the amino acid sequence of SEQ ID NO: 144; the VL CDR1 comprises the amino acid sequence of SEQ ID NO: 145; the VL CDR2 comprises the amino acid sequence of SEQ ID NO: 146; and the VL CDR3 comprises the amino acid sequence of SEQ ID NO: 147 or 148.

In some embodiments, the VH CDR1 comprises the amino acid sequence of SEQ ID NO: 149; the VH CDR2 comprises the amino acid sequence of SEQ ID NO: 150, 151 or 152; the VH CDR3 comprises the amino acid sequence of SEQ ID NO: 153 or 154; the VL CDR1 comprises the amino acid sequence of SEQ ID NO: 155; the VL CDR2 comprises the amino acid sequence of SEQ ID NO: 156; and the VL CDR3 comprises the amino acid sequence of SEQ ID NO: 157 or 158.

In some embodiments, the VH CDR1 comprises the amino acid sequence of SEQ ID NO: 149; the VH CDR2 comprises the amino acid sequence of SEQ ID NO: 150; the VH CDR3 comprises the amino acid sequence of SEQ ID NO: 153; the VL CDR1 comprises the amino acid sequence of SEQ ID NO: 155; the VL CDR2 comprises the amino acid sequence of SEQ ID NO: 156; and the VL CDR3 comprises the amino acid sequence of SEQ ID NO: 157.

In some embodiments, the VH comprises the amino acid sequence of SEQ ID NO: 118 and the VL comprises the amino acid sequence of SEQ ID NO: 119.

In some embodiments, the VH comprises the amino acid sequence of SEQ ID NO: 120 and the VL comprises the amino acid sequence of SEQ ID NO: 121.

In some embodiments, the VH comprises the amino acid sequence of SEQ ID NO: 122 and the VL comprises the amino acid sequence of SEQ ID NO: 123.

In some embodiments, the VH comprises the amino acid sequence of SEQ ID NO: 124 and the VL comprises the amino acid sequence of SEQ ID NO: 125.

In some embodiments, the antibody or fragment thereof is a bivalent Fab antibody, or a fragment selected from the group consisting of F(ab′)2, F(ab)2, Fab′, Fab, Fv, and scFv.

Also provided is a multispecific antibody comprising an antigen-binding fragment of the present disclosure and one or more antibody or antigen-binding fragment having binding specificity to a target antigen that is not B7-H3.

Still further provided is a chimeric antigen receptor (CAR) comprising an antigen-binding fragment of the present disclosure, a transmembrane domain, a costimulatory domain, and a CD3ξ intracellular domain. Yet further provided is a one or more polynucleotide(s) encoding the antibody or antigen-binding fragment thereof or the CAR of the present disclosure. In some embodiments, the polynucleotide is one or more mRNA.

Methods and uses for treating diseases such as cancer are also provided.

### BRIEF DESCRIPTION OF THE DRAWINGS

FIG. 1 shows that the tested antibodies bound to the B7-H3 protein on A375 tumor cells. Except for AHP05564, AHP06331, AHP06345, and AHP06358, all tested antibodies showed better binding than the clinical benchmark, MGA017.

FIG. 2 shows the internalization potency of tested antibodies on A375 tumor cells.

FIG. 3 shows the in vitro killing efficacy of B7-H3 ADCs against RKO tumor cells.

FIG. 4 shows the binding of humanized antibodies to the B7-H3 expressed on A375 tumor cells.

FIG. 5 shows the binding activity of chimeric antibodies after removal of potential PTM site against the B7-H3 expressed on A375 tumor cells.

FIG. 6 shows the binding activity of humanized antibodies with PTM removal to the A375 tumor cells.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” includes aptamers, spiegeleisen, and diabodies. The term “antibody fragment” also includes any synthetic or genetically engineered protein that acts like an antibody by binding to a specific antigen to form a complex.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively.

The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000 Daltons. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

Antibodies disclosed herein can be from any animal origin including birds and mammals. Preferably, the antibodies are human, murine, donkey, rabbit, goat, guinea pig, camel, llama, horse, or chicken antibodies. In some embodiments, the variable region may be condricthoid in origin (e.g., from sharks).

As used herein, the term “recombinant” as it pertains to polypeptides or polynucleotides intends a form of the polypeptide or polynucleotide that does not exist naturally, a non-limiting example of which can be created by combining polynucleotides that would not normally occur together.

Hybridoma technology can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A nonlimiting example of a physiological condition is the temperature, ionic strength, pH and concentration of Mg2+ normally found in a cell.

### Anti-B7-H3 Antibodies

As demonstrated in the appended experimental examples, the instant inventors were able to generate anti-B7-H3 antibodies 35A12B11, 62H9H5, 72D1D11, 81C8A1, 97E6B2, 106A5B3, 126C10B10, 216E7A9, 227E12D1, 294A3C4, 312E1E2, and 429H9F10 (Table 1) all of which have higher binding affinity to the human B7-H3 protein than the benchmark antibody MGA017 (MacroGenics).

Meanwhile, full human antibodies have been identified from a customized phage antibody library, including AHP05564, AHP06331, AHP06345 and AHP06358 (Table 2). These human antibodies also have unique properties to MGA017, underscoring the promise of these human antibodies.

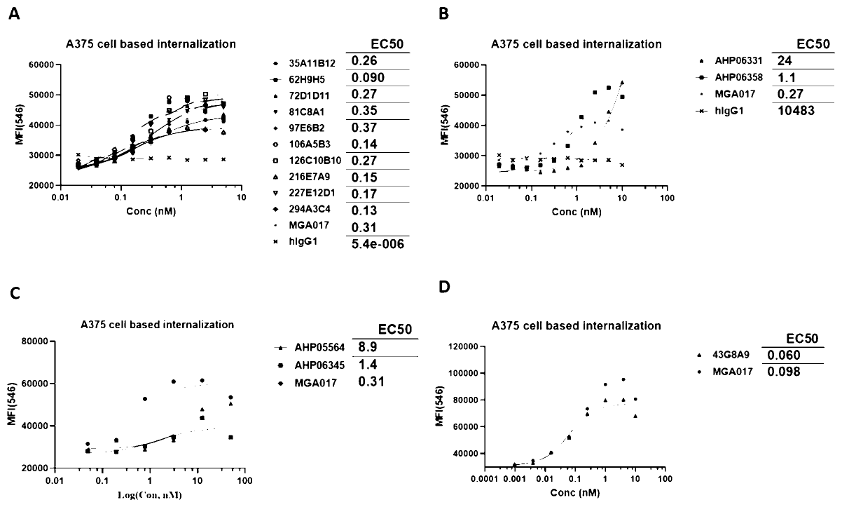

Likely attributable to the improved binding affinity, all of the chimeric versions of the murine antibodies resulted in higher cellular internalization than MGA017 when evaluated with A375 cells (FIG. 2A). Surprisingly, two of the full human antibodies, AHP06331 and AHP06358 also greatly outperformed MGA017 at high concentrations, despite their relatively modest performance at lower concentrations (FIG. 2B).

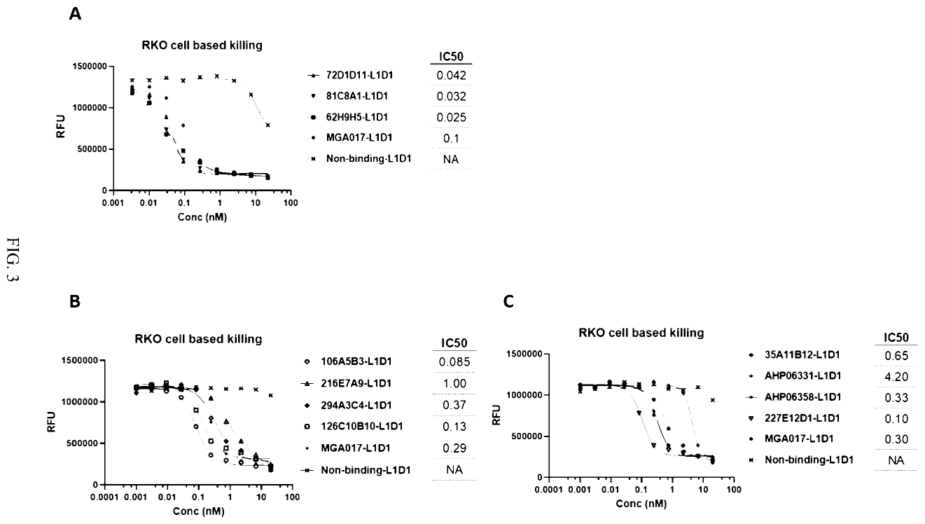

Antibody-drug conjugates (ADC) of these antibodies were also prepared and tested. Again, at least the ADC of 72D1D11, 81C8A1, 62H9H5, 106A5B3, 126C10B10, and 227E12D outperformed the vc-MMAE-conjugated MGA017 (FIG. 3), further substantiating the superior properties of these new antibodies.

Based on binding competition assays, the antibodies were categorized into five different epitope binning groups, Aa, AR, Ay, B and C, as shown in Table 3B. Group Aa includes MGA017, along with 62H9H5, 81C8A1, 106A5B3, 216E7A9, and 227E12D1, while most of the antibodies are not in this group. Group A3 includes 72D1D11, 97E6B2,126C10B10, and 429H9F10; Group Ay includes 35A12B11, 294A3C4, and AHP06358; Group B includes 312E1E12 and AHP06345; and Group C includes AHP06331 and AHP05564.

In accordance with one embodiment of the present disclosure, therefore, provided is an antibody or antigen-binding fragment thereof. In some embodiments, the antibody or antigen-binding fragment thereof has binding specificity to the human B7-H3 protein. In some embodiments, the antibody or antigen-binding fragment thereof includes a heavy chain variable region (VH) that includes a VH CDR1, a VH CDR2 and a VH CDR3, and a light chain variable region (VL) that includes a VL CDR1, a VL CDR2, and a VL CDR3.

In one embodiment, the VH CDR1, VH CDR2, VH CDR3, VL CDR1, VL CDR2, and VL CDR3 are those of any of the antibodies identified herein, such as 35A12B11, 62H9H5, 72D1D11, 81C8A1, 97E6B2, 106A5B3, 126C10B10, 216E7A9, 227E12D1, 294A3C4, 312E1E2, and 429H9F10, or any of AHP05564, AHP06331, AHP06345 and AHP06358.

In some embodiments, with reference to antibody 72D1D11, the VH CDR1 includes the amino acid sequence of SEQ ID NO: 39, the VH CDR2 includes the amino acid sequence of SEQ ID NO: 40, the VH CDR3 includes the amino acid sequence of SEQ ID NO: 41, the VL CDR1 includes the amino acid sequence of SEQ ID NO: 47, the VL CDR2 includes the amino acid sequence of SEQ ID NO: 50, and the VL CDR3 includes the amino acid sequence of SEQ ID NO: 51.

The antibody or fragment may be murine, chimeric, or humanized. Examples antibodies include those shown in Table 5. In some embodiments, the antibody or fragment has a VH having an amino acid sequence of SEQ ID NO: 5 (murine), 159, 160, 161 or 162 (humanized). In some embodiments, the antibody or fragment has a VL having an amino acid sequence of SEQ ID NO: 6 (murine), 163, 164, 165 or 166 (humanized). Example pairing of VH/VL sequences are shown in Table 5B. For instance, in one embodiment, the antibody or fragment thereof includes a VH of SEQ ID NO: 159 and VL of SEQ ID NO: 165. In another embodiment, the antibody or fragment thereof includes a VH of SEQ ID NO: 159 and VL of SEQ ID NO: 163.

Sequence analysis revealed that the VH CDR3 sequence (SEQ ID NO: 41) and VL CDR1 sequence (SEQ ID NO: 47) include residues that can potentially be modified post-translationally. To avoid post-translational modification (PTM) risks and thus simplify manufacturing, the instant disclosure designed and tested certain de-risked versions of the VH CDR3, including SEQ ID NO: 42, 43, 44, 45 and 46, and those of VL CDR1, including SEQ ID NO: 48 and 49.

In some embodiments, therefore, provided are antibodies and fragments having CDR sequences in which the VH CDR1 includes the amino acid sequence of SEQ ID NO: 39, the VH CDR2 includes the amino acid sequence of SEQ ID NO: 40, the VH CDR3 includes the amino acid sequence of SEQ ID NO: 41, 42, 43, 44, 45 or 46, the VL CDR1 includes the amino acid sequence of SEQ ID NO: 47, 48 or 49, the VL CDR2 includes the amino acid sequence of SEQ ID NO: 50, and the VL CDR3 includes the amino acid sequence of SEQ ID NO: 51.

In some embodiments, the VH CDR1 includes the amino acid sequence of SEQ ID NO: 39, the VH CDR2 includes the amino acid sequence of SEQ ID NO: 40, the VH CDR3 includes the amino acid sequence of SEQ ID NO: 41, the VL CDR1 includes the amino acid sequence of SEQ ID NO: 47, 48 or 49, the VL CDR2 includes the amino acid sequence of SEQ ID NO: 50, and the VL CDR3 includes the amino acid sequence of SEQ ID NO: 51.

Example antibody sequences with or without the PTM-derisked CDRs are also provided. As shown in Table 9. In some embodiments, the antibody or fragment has a VH having an amino acid sequence of SEQ ID NO: 159, 174, 175, 176, or 177. In some embodiments, the antibody or fragment has a VL having an amino acid sequence of SEQ ID NO: 165, 178 or 179. Example pairing of VH/VL sequences are shown in Table 8. For instance, in one embodiment, the antibody or fragment thereof includes a VH of SEQ ID NO: 159 and VL of SEQ ID NO: 178. In another embodiment, the antibody or fragment thereof includes a VH of SEQ ID NO: 159 and VL of SEQ ID NO: 179.

Also provided, in some embodiments, are antibodies and antigen-binding fragments therefore that bind to the same epitope on B7-H3 as 72D1D11. Also provided, in some embodiments, are antibodies and antigen-binding fragments therefore that compete with 72D1D11 in binding to B7-H3.

In some embodiments, with reference to antibody 81C8A1, the VH CDR1 includes the amino acid sequence of SEQ ID NO: 52, the VH CDR2 includes the amino acid sequence of SEQ ID NO: 53, the VH CDR3 includes the amino acid sequence of SEQ ID NO: 54, the VL CDR1 includes the amino acid sequence of SEQ ID NO: 55, the VL CDR2 includes the amino acid sequence of SEQ ID NO: 56, and the VL CDR3 includes the amino acid sequence of SEQ ID NO: 57.

The antibody or fragment may be murine, chimeric, or humanized. Examples antibodies include those shown in Table 6. In some embodiments, the antibody or fragment has a VH having an amino acid sequence of SEQ ID NO: 7 (murine), 167, 168 or 169 (humanized). In some embodiments, the antibody or fragment has a VL having an amino acid sequence of SEQ ID NO: 8 (murine), 170, 171, 172 or 173 (humanized). Example pairing of VH/VL sequences are shown in Table 6B. For instance, in one embodiment, the antibody or fragment thereof includes a VH of SEQ ID NO: 167 and VL of SEQ ID NO: 172. In another embodiment, the antibody or fragment thereof includes a VH of SEQ ID NO: 167 and VL of SEQ ID NO: 173. In another embodiment, the antibody or fragment thereof includes a VH of SEQ ID NO: 168 and VL of SEQ ID NO: 171. In another embodiment, the antibody or fragment thereof includes a VH of SEQ ID NO: 168 and VL of SEQ ID NO: 172. In another embodiment, the antibody or fragment thereof includes a VH of SEQ ID NO: 168 and VL of SEQ ID NO: 173.

Also provided, in some embodiments, are antibodies and antigen-binding fragments therefore that bind to the same epitope on B7-H3 as 81C8A1. Also provided, in some embodiments, are antibodies and antigen-binding fragments therefore that compete with 81C8A1 in binding to B7-H3.

In some embodiments, with reference to antibody 35A12B11, the VH CDR1 includes the amino acid sequence of SEQ ID NO: 25, the VH CDR2 includes the amino acid sequence of SEQ ID NO: 26, the VH CDR3 includes the amino acid sequence of SEQ ID NO: 29, the VL CDR1 includes the amino acid sequence of SEQ ID NO: 30, the VL CDR2 includes the amino acid sequence of SEQ ID NO: 31, and the VL CDR3 includes the amino acid sequence of SEQ ID NO: 32.

Sequence analysis revealed that the VH CDR2 sequence (SEQ ID NO: 26) includes residues that can potentially be modified post-translationally. To avoid post-translational modification (PTM) risks and thus simplify manufacturing, the instant disclosure designed and tested certain de-risked versions of the VH CDR2, including SEQ ID NO: 27 and 28.

In some embodiments, therefore, provided are antibodies and fragments having CDR sequences in which the VH CDR1 includes the amino acid sequence of SEQ ID NO: 25, the VH CDR2 includes the amino acid sequence of SEQ ID NO: 26, 27 or 28, the VH CDR3 includes the amino acid sequence of SEQ ID NO: 29, the VL CDR1 includes the amino acid sequence of SEQ ID NO: 30, the VL CDR2 includes the amino acid sequence of SEQ ID NO: 31, and the VL CDR3 includes the amino acid sequence of SEQ ID NO: 32.

In one embodiment, the antibody or fragment thereof includes a VH of SEQ ID NO: 1 and VL of SEQ ID NO: 2. Also provided, in some embodiments, are antibodies and antigen-binding fragments therefore that bind to the same epitope on B7-H3 as 35A12B11. Also provided, in some embodiments, are antibodies and antigen-binding fragments therefore that compete with 35A12B11 in binding to B7-H3.

In some embodiments, with reference to antibody 62H9H5, the VH CDR1 includes the amino acid sequence of SEQ ID NO: 33, the VH CDR2 includes the amino acid sequence of SEQ ID NO: 34, the VH CDR3 includes the amino acid sequence of SEQ ID NO: 35, the VL CDR1 includes the amino acid sequence of SEQ ID NO: 36, the VL CDR2 includes the amino acid sequence of SEQ ID NO: 37, and the VL CDR3 includes the amino acid sequence of SEQ ID NO: 38.

In one embodiment, the antibody or fragment thereof includes a VH of SEQ ID NO: 3 and VL of SEQ ID NO: 4. Also provided, in some embodiments, are antibodies and antigen-binding fragments therefore that bind to the same epitope on B7-H3 as 62H9H5. Also provided, in some embodiments, are antibodies and antigen-binding fragments therefore that compete with 62H9H5 in binding to B7-H3.

In some embodiments, with reference to antibody 97E6B2, the VH CDR1 includes the amino acid sequence of SEQ ID NO: 58, the VH CDR2 includes the amino acid sequence of SEQ ID NO: 59, the VH CDR3 includes the amino acid sequence of SEQ ID NO: 60, the VL CDR1 includes the amino acid sequence of SEQ ID NO: 61, the VL CDR2 includes the amino acid sequence of SEQ ID NO: 62, and the VL CDR3 includes the amino acid sequence of SEQ ID NO: 63.

In one embodiment, the antibody or fragment thereof includes a VH of SEQ ID NO: 9 and VL of SEQ ID NO: 10. Also provided, in some embodiments, are antibodies and antigen-binding fragments therefore that bind to the same epitope on B7-H3 as 97E6B2. Also provided, in some embodiments, are antibodies and antigen-binding fragments therefore that compete with 97E6B2 in binding to B7-H3.

In some embodiments, with reference to antibody 106A5B3, the VH CDR1 includes the amino acid sequence of SEQ ID NO: 64, the VH CDR2 includes the amino acid sequence of SEQ ID NO: 65, the VH CDR3 includes the amino acid sequence of SEQ ID NO: 67, the VL CDR1 includes the amino acid sequence of SEQ ID NO: 68, the VL CDR2 includes the amino acid sequence of SEQ ID NO: 69, and the VL CDR3 includes the amino acid sequence of SEQ ID NO: 70.

Sequence analysis revealed that the VH CDR2 sequence (SEQ ID NO: 65) includes residues that can potentially be modified post-translationally. To avoid post-translational modification (PTM) risks and thus simplify manufacturing, the instant disclosure designed and tested certain de-risked version of the VH CDR2, including SEQ ID NO: 66.

In some embodiments, therefore, provided are antibodies and fragments having CDR sequences in which the VH CDR1 includes the amino acid sequence of SEQ ID NO: 64, the VH CDR2 includes the amino acid sequence of SEQ ID NO: 65 or 66, the VH CDR3 includes the amino acid sequence of SEQ ID NO: 67, the VL CDR1 includes the amino acid sequence of SEQ ID NO: 68, the VL CDR2 includes the amino acid sequence of SEQ ID NO: 69, and the VL CDR3 includes the amino acid sequence of SEQ ID NO: 70.

In one embodiment, the antibody or fragment thereof includes a VH of SEQ ID NO: 11 and VL of SEQ ID NO: 12. Also provided, in some embodiments, are antibodies and antigen-binding fragments therefore that bind to the same epitope on B7-H3 as 106A5B3. Also provided, in some embodiments, are antibodies and antigen-binding fragments therefore that compete with 106A5B3 in binding to B7-H3.

In some embodiments, with reference to antibody 126C10B10, the VH CDR1 includes the amino acid sequence of SEQ ID NO: 71, the VH CDR2 includes the amino acid sequence of SEQ ID NO: 72, the VH CDR3 includes the amino acid sequence of SEQ ID NO: 75, the VL CDR1 includes the amino acid sequence of SEQ ID NO: 76, the VL CDR2 includes the amino acid sequence of SEQ ID NO: 77, and the VL CDR3 includes the amino acid sequence of SEQ ID NO: 78.

Sequence analysis revealed that the VH CDR2 sequence (SEQ ID NO: 72) includes residues that can potentially be modified post-translationally. To avoid post-translational modification (PTM) risks and thus simplify manufacturing, the instant disclosure designed and tested certain de-risked versions of the VH CDR2, including SEQ ID NO: 73 and 74.

In some embodiments, therefore, provided are antibodies and fragments having CDR sequences in which the VH CDR1 includes the amino acid sequence of SEQ ID NO: 71, the VH CDR2 includes the amino acid sequence of SEQ ID NO: 72, 73 or 74, the VH CDR3 includes the amino acid sequence of SEQ ID NO: 75, the VL CDR1 includes the amino acid sequence of SEQ ID NO: 76, the VL CDR2 includes the amino acid sequence of SEQ ID NO: 77, and the VL CDR3 includes the amino acid sequence of SEQ ID NO: 78.

In one embodiment, the antibody or fragment thereof includes a VH of SEQ ID NO: 13 and VL of SEQ ID NO: 14. Also provided, in some embodiments, are antibodies and antigen-binding fragments therefore that bind to the same epitope on B7-H3 as 126C10B10. Also provided, in some embodiments, are antibodies and antigen-binding fragments therefore that compete with 126C10B10 in binding to B7-H3.

In some embodiments, with reference to antibody 216E7A9, the VH CDR1 includes the amino acid sequence of SEQ ID NO: 79, the VH CDR2 includes the amino acid sequence of SEQ ID NO: 80, the VH CDR3 includes the amino acid sequence of SEQ ID NO: 81, the VL CDR1 includes the amino acid sequence of SEQ ID NO: 82, the VL CDR2 includes the amino acid sequence of SEQ ID NO: 83, and the VL CDR3 includes the amino acid sequence of SEQ ID NO: 84.

In one embodiment, the antibody or fragment thereof includes a VH of SEQ ID NO: 15 and VL of SEQ ID NO: 16. Also provided, in some embodiments, are antibodies and antigen-binding fragments therefore that bind to the same epitope on B7-H3 as 216E7A9. Also provided, in some embodiments, are antibodies and antigen-binding fragments therefore that compete with 216E7A9 in binding to B7-H3.

In some embodiments, with reference to antibody 227E12D1, the VH CDR1 includes the amino acid sequence of SEQ ID NO: 85, the VH CDR2 includes the amino acid sequence of SEQ ID NO: 86, the VH CDR3 includes the amino acid sequence of SEQ ID NO: 88, the VL CDR1 includes the amino acid sequence of SEQ ID NO: 89, the VL CDR2 includes the amino acid sequence of SEQ ID NO: 90, and the VL CDR3 includes the amino acid sequence of SEQ ID NO: 91.

Sequence analysis revealed that the VH CDR2 sequence (SEQ ID NO: 86) includes residues that can potentially be modified post-translationally. To avoid post-translational modification (PTM) risks and thus simplify manufacturing, the instant disclosure designed and tested certain de-risked version of the VH CDR2, including SEQ ID NO: 87.

In some embodiments, therefore, provided are antibodies and fragments having CDR sequences in which the VH CDR1 includes the amino acid sequence of SEQ ID NO: 85, the VH CDR2 includes the amino acid sequence of SEQ ID NO: 86 or 87, the VH CDR3 includes the amino acid sequence of SEQ ID NO: 88, the VL CDR1 includes the amino acid sequence of SEQ ID NO: 89, the VL CDR2 includes the amino acid sequence of SEQ ID NO: 90, and the VL CDR3 includes the amino acid sequence of SEQ ID NO: 91.

In one embodiment, the antibody or fragment thereof includes a VH of SEQ ID NO: 17 and VL of SEQ ID NO: 18. Also provided, in some embodiments, are antibodies and antigen-binding fragments therefore that bind to the same epitope on B7-H3 as 227E12D1. Also provided, in some embodiments, are antibodies and antigen-binding fragments therefore that compete with 227E12D1 in binding to B7-H3.

In some embodiments, with reference to antibody 294A3C4, the VH CDR1 includes the amino acid sequence of SEQ ID NO: 92, the VH CDR2 includes the amino acid sequence of SEQ ID NO: 93, the VH CDR3 includes the amino acid sequence of SEQ ID NO: 97, the VL CDR1 includes the amino acid sequence of SEQ ID NO: 98, the VL CDR2 includes the amino acid sequence of SEQ ID NO: 99, and the VL CDR3 includes the amino acid sequence of SEQ ID NO: 101.

Sequence analysis revealed that the VH CDR2 sequence (SEQ ID NO: 93) and VL CDR2 (SEQ ID NO:99) include residues that can potentially be modified post-translationally. To avoid post-translational modification (PTM) risks and thus simplify manufacturing, the instant disclosure designed and tested certain de-risked version of the VH CDR2, including SEQ ID NO: 94, 95, and 96, and that of VL CDR2, including SEQ ID NO: 100.

In some embodiments, therefore, provided are antibodies and fragments having CDR sequences in which the VH CDR1 includes the amino acid sequence of SEQ ID NO: 92, the VH CDR2 includes the amino acid sequence of SEQ ID NO: 93, 94, 95 or 96, the VH CDR3 includes the amino acid sequence of SEQ ID NO: 97, the VL CDR1 includes the amino acid sequence of SEQ ID NO: 98, the VL CDR2 includes the amino acid sequence of SEQ ID NO: 99 or 100, and the VL CDR3 includes the amino acid sequence of SEQ ID NO: 101.

In one embodiment, the antibody or fragment thereof includes a VH of SEQ ID NO: 19 and VL of SEQ ID NO: 20. Also provided, in some embodiments, are antibodies and antigen-binding fragments therefore that bind to the same epitope on B7-H3 as 294A3C4. Also provided, in some embodiments, are antibodies and antigen-binding fragments therefore that compete with 294A3C4 in binding to B7-H3.

In some embodiments, with reference to antibody 312E1E2, the VH CDR1 includes the amino acid sequence of SEQ ID NO: 102, the VH CDR2 includes the amino acid sequence of SEQ ID NO: 103, the VH CDR3 includes the amino acid sequence of SEQ ID NO: 104, the VL CDR1 includes the amino acid sequence of SEQ ID NO: 105, the VL CDR2 includes the amino acid sequence of SEQ ID NO: 106, and the VL CDR3 includes the amino acid sequence of SEQ ID NO: 107.

In one embodiment, the antibody or fragment thereof includes a VH of SEQ ID NO: 21 and VL of SEQ ID NO: 22. Also provided, in some embodiments, are antibodies and antigen-binding fragments therefore that bind to the same epitope on B7-H3 as 312E1E2. Also provided, in some embodiments, are antibodies and antigen-binding fragments therefore that compete with 312E1E2 in binding to B7-H3.

In some embodiments, with reference to antibody 429H9F10, the VH CDR1 includes the amino acid sequence of SEQ ID NO: 108, the VH CDR2 includes the amino acid sequence of SEQ ID NO: 109, the VH CDR3 includes the amino acid sequence of SEQ ID NO: 110, the VL CDR1 includes the amino acid sequence of SEQ ID NO: 113, the VL CDR2 includes the amino acid sequence of SEQ ID NO: 116, and the VL CDR3 includes the amino acid sequence of SEQ ID NO: 117.

Sequence analysis revealed that the VH CDR3 sequence (SEQ ID NO: 110) and VL CDR1 (SEQ ID NO:113) include residues that can potentially be modified post-translationally. To avoid post-translational modification (PTM) risks and thus simplify manufacturing, the instant disclosure designed and tested certain de-risked version of the VH CDR3, including SEQ ID NO: 111 and 112, and that of VL CDR1, including SEQ ID NO: 114 and 115.

In some embodiments, therefore, provided are antibodies and fragments having CDR sequences in which the VH CDR1 includes the amino acid sequence of SEQ ID NO: 108, the VH CDR2 includes the amino acid sequence of SEQ ID NO: 109, the VH CDR3 includes the amino acid sequence of SEQ ID NO: 110, 111 or 112, the VL CDR1 includes the amino acid sequence of SEQ ID NO: 113, 114 or 115, the VL CDR2 includes the amino acid sequence of SEQ ID NO: 116, and the VL CDR3 includes the amino acid sequence of SEQ ID NO: 117.

In one embodiment, the antibody or fragment thereof includes a VH of SEQ ID NO: 23 and VL of SEQ ID NO: 24. Also provided, in some embodiments, are antibodies and antigen-binding fragments therefore that bind to the same epitope on B7-H3 as 429H9F10. Also provided, in some embodiments, are antibodies and antigen-binding fragments therefore that compete with 429H9F10 in binding to B7-H3.

In some embodiments, with reference to antibody 43G8A9, the VH CDR1 includes the amino acid sequence of SEQ ID NO: 182; the VH CDR2 includes the amino acid sequence of SEQ ID NO: 183; the VH CDR3 includes the amino acid sequence of SEQ ID NO: 187; the VL CDR1 includes the amino acid sequence of SEQ ID NO: 188; the VL CDR2 includes the amino acid sequence of SEQ ID NO: 192; and the VL CDR3 includes the amino acid sequence of SEQ ID NO: 193.

Sequence analysis revealed that the VH CDR2 sequence (SEQ ID NO: 183) and VL CDR1 (SEQ ID NO:188) include residues that can potentially be modified post-translationally. To avoid post-translational modification (PTM) risks and thus simplify manufacturing, the instant disclosure designed and tested certain de-risked version of the VH CDR2, including SEQ ID NO: 184, 185 and 186, and that of VL CDR1, including SEQ ID NO: 188, 189, 190 and 191.

In some embodiments, therefore, provided are antibodies and fragments having CDR sequences in which the VH CDR1 includes the amino acid sequence of SEQ ID NO: 182; the VH CDR2 includes the amino acid sequence of SEQ ID NO: 183, 184, 185, or 186; the VH CDR3 includes the amino acid sequence of SEQ ID NO: 187; the VL CDR1 includes the amino acid sequence of SEQ ID NO: 188, 189, 190, or 191; the VL CDR2 includes the amino acid sequence of SEQ ID NO: 192; and the VL CDR3 includes the amino acid sequence of SEQ ID NO: 193.

In one embodiment, the antibody or fragment thereof includes a VH of SEQ ID NO: 180 and VL of SEQ ID NO: 181. Also provided, in some embodiments, are antibodies and antigen-binding fragments therefore that bind to the same epitope on B7-H3 as 43G8A9. Also provided, in some embodiments, are antibodies and antigen-binding fragments therefore that compete with 43G8A9 in binding to B7-H3.

In some embodiments, with reference to antibody AHP05564, the VH CDR1 includes the amino acid sequence of SEQ ID NO: 126, the VH CDR2 includes the amino acid sequence of SEQ ID NO: 127, the VH CDR3 includes the amino acid sequence of SEQ ID NO: 129, the VL CDR1 includes the amino acid sequence of SEQ ID NO: 132, the VL CDR2 includes the amino acid sequence of SEQ ID NO: 133, and the VL CDR3 includes the amino acid sequence of SEQ ID NO: 134.

Sequence analysis revealed that the VH CDR2 (SEQ ID NO:127) and VH CDR3 sequence (SEQ ID NO: 129) include residues that can potentially be modified post-translationally. To avoid post-translational modification (PTM) risks and thus simplify manufacturing, the instant disclosure designed and tested certain de-risked version of the VH CDR2, including SEQ ID NO: 128, and that of VH CDR3, including SEQ ID NO: 130 and 131.

In some embodiments, therefore, provided are antibodies and fragments having CDR sequences in which the VH CDR1 includes the amino acid sequence of SEQ ID NO: 126, the VH CDR2 includes the amino acid sequence of SEQ ID NO: 127 or 128, the VH CDR3 includes the amino acid sequence of SEQ ID NO: 129, 130 or 131, the VL CDR1 includes the amino acid sequence of SEQ ID NO: 132, the VL CDR2 includes the amino acid sequence of SEQ ID NO: 133, and the VL CDR3 includes the amino acid sequence of SEQ ID NO: 134.

In one embodiment, the antibody or fragment thereof includes a VH of SEQ ID NO: 118 and VL of SEQ ID NO: 119. Also provided, in some embodiments, are antibodies and antigen-binding fragments therefore that bind to the same epitope on B7-H3 as AHP05564. Also provided, in some embodiments, are antibodies and antigen-binding fragments therefore that compete with AHP05564 in binding to B7-H3.

In some embodiments, with reference to antibody AHP06331, the VH CDR1 includes the amino acid sequence of SEQ ID NO: 135, the VH CDR2 includes the amino acid sequence of SEQ ID NO: 136, the VH CDR3 includes the amino acid sequence of SEQ ID NO: 138, the VL CDR1 includes the amino acid sequence of SEQ ID NO: 139, the VL CDR2 includes the amino acid sequence of SEQ ID NO: 140, and the VL CDR3 includes the amino acid sequence of SEQ ID NO: 141.

Sequence analysis revealed that the VH CDR2 (SEQ ID NO: 136) includes residues that can potentially be modified post-translationally. To avoid post-translational modification (PTM) risks and thus simplify manufacturing, the instant disclosure designed and tested certain de-risked version of the VH CDR2, including SEQ ID NO: 137.

In some embodiments, therefore, provided are antibodies and fragments having CDR sequences in which the VH CDR1 includes the amino acid sequence of SEQ ID NO: 135, the VH CDR2 includes the amino acid sequence of SEQ ID NO: 136 or 137, the VH CDR3 includes the amino acid sequence of SEQ ID NO: 138, the VL CDR1 includes the amino acid sequence of SEQ ID NO: 139, the VL CDR2 includes the amino acid sequence of SEQ ID NO: 140, and the VL CDR3 includes the amino acid sequence of SEQ ID NO: 141.

In one embodiment, the antibody or fragment thereof includes a VH of SEQ ID NO: 120 and VL of SEQ ID NO: 121. Also provided, in some embodiments, are antibodies and antigen-binding fragments therefore that bind to the same epitope on B7-H3 as AHP06331. Also provided, in some embodiments, are antibodies and antigen-binding fragments therefore that compete with AHP06331 in binding to B7-H3.

In some embodiments, with reference to antibody AHP06345, the VH CDR1 includes the amino acid sequence of SEQ ID NO: 142, the VH CDR2 includes the amino acid sequence of SEQ ID NO: 143, the VH CDR3 includes the amino acid sequence of SEQ ID NO: 144, the VL CDR1 includes the amino acid sequence of SEQ ID NO: 145, the VL CDR2 includes the amino acid sequence of SEQ ID NO: 146, and the VL CDR3 includes the amino acid sequence of SEQ ID NO: 147.

Sequence analysis revealed that the VL CDR3 (SEQ ID NO:147) includes residues that can potentially be modified post-translationally. To avoid post-translational modification (PTM) risks and thus simplify manufacturing, the instant disclosure designed and tested certain de-risked version of the VL CDR3, including SEQ ID NO: 148.

In some embodiments, therefore, provided are antibodies and fragments having CDR sequences in which the VH CDR1 includes the amino acid sequence of SEQ ID NO: 142, the VH CDR2 includes the amino acid sequence of SEQ ID NO: 143, the VH CDR3 includes the amino acid sequence of SEQ ID NO: 144, the VL CDR1 includes the amino acid sequence of SEQ ID NO: 145, the VL CDR2 includes the amino acid sequence of SEQ ID NO: 146, and the VL CDR3 includes the amino acid sequence of SEQ ID NO: 147 or 148.

In one embodiment, the antibody or fragment thereof includes a VH of SEQ ID NO: 122 and VL of SEQ ID NO: 123. Also provided, in some embodiments, are antibodies and antigen-binding fragments therefore that bind to the same epitope on B7-H3 as AHP06345. Also provided, in some embodiments, are antibodies and antigen-binding fragments therefore that compete with AHP06345 in binding to B7-H3.

In some embodiments, with reference to antibody AHP06358, the VH CDR1 includes the amino acid sequence of SEQ ID NO: 149, the VH CDR2 includes the amino acid sequence of SEQ ID NO: 150, the VH CDR3 includes the amino acid sequence of SEQ ID NO: 153, the VL CDR1 includes the amino acid sequence of SEQ ID NO: 155, the VL CDR2 includes the amino acid sequence of SEQ ID NO: 156, and the VL CDR3 includes the amino acid sequence of SEQ ID NO: 157.

Sequence analysis revealed that the VH CDR2 (SEQ ID NO: 150), VH CDR3 (SEQ ID NO:153) and VL CDR3 (SEQ ID NO: 157) includes residues that can potentially be modified post-translationally. To avoid post-translational modification (PTM) risks and thus simplify manufacturing, the instant disclosure designed and tested certain de-risked version of the VH CDR2, including SEQ ID NO: 151 and 152, that of VH CDR3 including SEQ ID NO: 154, and that of VL CDR3 including SEQ ID NO: 158.

In some embodiments, therefore, provided are antibodies and fragments having CDR sequences in which the VH CDR1 includes the amino acid sequence of SEQ ID NO: 149, the VH CDR2 includes the amino acid sequence of SEQ ID NO: 150, 151 or 152, the VH CDR3 includes the amino acid sequence of SEQ ID NO: 153 or 154, the VL CDR1 includes the amino acid sequence of SEQ ID NO: 155, the VL CDR2 includes the amino acid sequence of SEQ ID NO: 156, and the VL CDR3 includes the amino acid sequence of SEQ ID NO: 157 or 158.

In one embodiment, the antibody or fragment thereof includes a VH of SEQ ID NO: 124 and VL of SEQ ID NO: 125. Also provided, in some embodiments, are antibodies and antigen-binding fragments therefore that bind to the same epitope on B7-H3 as AHP06358. Also provided, in some embodiments, are antibodies and antigen-binding fragments therefore that compete with AHP06358 in binding to B7-H3.

Also provided, in some embodiments, are antibodies and antigen-binding fragments that include CDR sequences derived from the presently disclosed CDR sequences, with one, two or three amino acid substitutions, deletions, and/or additions.

In some embodiments, the anti-B7-H3 antibodies are modified mAbs comprising a modified heavy chain constant region, such as an afucosylated heavy chain, that binds with higher affinity to activating Fcγ receptor that mediated enhanced ADCC compared to an unmodified mAb. In some embodiments, the anti-B7-H3 antibodies comprises a heavy chain which is of a human IgG1 variant that include the single or combination of L234Y, L235Q, G236W, S239D/M, F243L, H268D, D270E, R292P, S298A, Y300L, V305I, K326D, A330L/M, I332E, K334A/E, P396L that enhance ADCC function (all EU numbering).

### Antibody-Drug Conjugates

In vitro and in vivo data show that antibody-drug conjugates (ADC) derived from the new antibodies had higher anti-tumor activity then the benchmark antibody MGA017, making them suitable for use in ADCs.

In some embodiments, the antibodies or fragments may be conjugated to therapeutic agents, prodrugs, peptides, proteins, enzymes, viruses, lipids, biological response modifiers, pharmaceutical agents, or PEG.

In one embodiment, the antibodies or fragments of the disclosure are covalently attached to a drug moiety. The drug moiety may be, or be modified to include, a group reactive with a conjugation point on the antibody. For example, a drug moiety can be attached by alkylation (e.g., at the epsilon-amino group lysines or the N-terminus of antibodies), reductive amination of oxidized carbohydrate, transesterification between hydroxyl and carboxyl groups, amidation at amino groups or carboxyl groups, and conjugation to thiols.

In some embodiments, the number of drug moieties, p, conjugated per antibody molecule ranges from an average of 1 to 8; 1 to 7, 1 to 6, 1 to 5, 1 to 4, 1 to 3, or 1 to 2. In some embodiments, p ranges from an average of 2 to 8, 2 to 7, 2 to 6, 2 to 5, 2 to 4 or 2 to 3. In other embodiments, p is an average of 1, 2, 3, 4, 5, 6, 7 or 8. In some embodiments, p ranges from an average of about 1 to about 20, about 1 to about 10, about 2 to about 10, about 2 to about 9, about 1 to about 8, about 1 to about 7, about 1 to about 6, about 1 to about 5, about 1 to about 4, about 1 to about 3, or about 1 to about 2. In some embodiments, p ranges from about 2 to about 8, about 2 to about 7, about 2 to about 6, about 2 to about 5, about 2 to about 4 or about 2 to about 3.

For example, when chemical activation of the protein results in formation of free thiol groups, the protein may be conjugated with a sulfhydryl reactive agent. In one aspect, the agent is one which is substantially specific for free thiol groups. Such agents include, for example, malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio.

The drug can be linked to the antibody or fragment by a linker. Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (me-Val-Cit-PABA) linker. Another linker is Sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Yet another linker is maleimidocaproyl (mc). Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the me linker and the like.

A linker can include a group for linkage to the antibody. For example, linker can include an amino, hydroxyl, carboxyl or sulfhydryl reactive groups (e.g., malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio).

In some embodiments, the drug moiety is a cytotoxic or cytostatic agent, an immunosuppressive agent, a radioisotope, a toxin, or the like. The conjugate can be used for inhibiting the multiplication of a tumor cell or cancer cell, causing apoptosis in a tumor or cancer cell, or for treating cancer in a patient. The conjugate can be used accordingly in a variety of settings for the treatment of animal cancers. The conjugate can be used to deliver a drug to a tumor cell or cancer cell. Without being bound by theory, in some embodiments, the conjugate binds to or associates with a cancer cell expressing GPC3, and the conjugate and/or drug can be taken up inside a tumor cell or cancer cell through receptor-mediated endocytosis.

Once inside the cell, one or more specific peptide sequences within the conjugate (e.g., in a linker) are hydrolytically cleaved by one or more tumor-cell or cancer-cell-associated proteases, resulting in release of the drug. The released drug is then free to migrate within the cell and induce cytotoxic or cytostatic or other activities. In some embodiments, the drug is cleaved from the antibody outside the tumor cell or cancer cell, and the drug subsequently penetrates the cell, or acts at the cell surface.

Examples of drug moieties or payloads are selected from the group consisting of DM1 (maytansine, N2′-deacetyl-N2′-(3-mercapto-1-oxopropyl)- or N2′-deacetyl-N2′-(3-mercapto-1-oxopropyl)-maytansine), me-MMAD (6-maleimidocaproyl-monomethylauristatin-D or N-methyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyr rolidinyl]-1-[(1S)-1-methylpropyl]-4-oxobutyl]-N-methyl-(9C1)-L-valinamide), me-MMAF (maleimidocaproyl-monomethylauristatin F or N-[6-(2,5-dihydro-2,5-dioxo-1H-pyrrol-1-yl)-1-oxohexyl]-N-methyl-L-valyl-L-valyl-(3R,4S,5S)-3-methoxy-5-methyl-4-(methylamino)heptanoyl-(αR, βR,2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[[4-[[N-[6-(2,5-dihydro-2,5-dioxo-1H-pyrrol-1-yl)-1-oxohexyl]-L-valyl-N5-(aminocarbonyl)-L-ornithyl]amino]phenyl]methoxy]carbonyl]-N-meth yl-L-valyl-N-[(1S,2R)-4-[(2S)-2-[(1R,2R)-3-[[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of the tubulin inhibitor maytansine while MMAD, MMAE, and MMAF are auristatin derivatives. In some embodiments, the drug moiety is selected from the group consisting of me-MMAF and mc-Val-Cit-PABA-MMAE. In some embodiments, the drug moiety is a maytansinoid or an auristatin.

The antibodies or fragments may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Multi-Functional Molecules

Multi-functional molecules that include an antibody or antigen-binding fragment specific to B7-H3, such as those disclosed herein, and one or more antibody or antigen-binding fragment having specificity to a second antigen.

In some embodiments, the second antigen is a protein expressed on an immune cell, such as a T cell, a B cell, a monocyte, a macrophage, a neutrophil, a dendritic cell, a phagocyte, a natural killer cell, an eosinophil, a basophil, and a mast cell.

In some embodiments, the second antigen is CD3, CD47, PD1, PD-L1, LAG3, TIM3, CTLA4, VISTA, CSFR1, A2AR, CD73, CD39, CD40, CEA, HER2, CMET, 4-1BB, OX40, SIRPA CD16, CD28, ICOS, CTLA4, BTLA, TIGIT, HVEM, CD27, VEGFR, or VEGF.

Different formats of bispecific antibodies are also provided. In some embodiments, each of the anti-B7-H3 fragment and the second fragment each is independently selected from a Fab fragment, a single-chain variable fragment (scFv), or a single-domain antibody. In some embodiments, the bispecific antibody further includes a Fc fragment.

Bifunctional molecules that include not just antibody or antigen binding fragment are also provided. As a tumor antigen targeting molecule, an antibody or antigen-binding fragment specific to B7-H3, such as those described here, can be combined with an immune cytokine or ligand optionally through a peptide linker. The linked immune cytokines or ligands include, but not limited to, IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL. Such bifunctional molecules can combine the immune checkpoint blocking effect with tumor site local immune modulation.

### Chimeric Antigen Receptors

Also provided, in one embodiment, is a chimeric antigen receptor (CAR) that includes the antibody or fragment thereof of the present disclosure as a targeting unit. In some embodiments, the CAR includes an antibody or fragment thereof of the present disclosure, a transmembrane domain, a costimulatory domain, and a CD3 intracellular domain.

A transmembrane domain can be designed to be fused to the extracellular domain which includes the antibody or fragment, optionally through a hinge domain. It can similarly be fused to an intracellular domain, such as a costimulatory domain. In some embodiments, the transmembrane domain can include the natural transmembrane region of a costimulatory domain (e.g., the TM region of a CD28T or 4-1BB employed as a costimulatory domain) or the natural transmembrane domain of a hinge region (e.g., the TM region of a CD8 alpha or CD28T employed as a hinge domain).

In some embodiments, the transmembrane domain can include a sequence that spans a cell membrane, but extends into the cytoplasm of a cell and/or into the extracellular space. For example, a transmembrane can include a membrane-spanning sequence which itself can further include 1, 2, 3, 4, 5, 6, 7, 8, 9, 10 or more amino acids that extend into the cytoplasm of a cell, and/or the extracellular space. Thus, a transmembrane domain includes a membrane-spanning region, yet can further comprise an amino acid(s) that extend beyond the internal or external surface of the membrane itself; such sequences can still be considered to be a “transmembrane domain”.

In some embodiments, the transmembrane domain is fused to the cytoplasmic domain through a short linker. Optionally, the short peptide or polypeptide linker, preferably between 2 and 10 amino acids in length can form the linkage between the transmembrane domain and a proximal cytoplasmic signaling domain of the chimeric receptor. A glycine-serine doublet (GS), glycine-serine-glycine triplet (GSG), or alanine-alanine-alanine triplet (AAA) provides a suitable linker.

In some embodiments, the CAR further includes a costimulatory domain. In some embodiments, the costimulatory domain is positioned between the transmembrane domain and an activating domain. Example costimulatory domains include, but are not limited to, CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD18 (ITGB2), CD19 (B4), CD27 (T FRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD 100 (SEMA4D), CD 103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KTR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (TNFRSF 18), inducible T cell co-stimulator (ICOS), LFA-1 (CD 1 1a/CD 18), KG2C, DAP-10, ICAM-1, Kp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

In some embodiments, the cytoplasmic portion of the CAR also includes a signaling/activation domain. In one embodiment, the signaling/activation domain is the CD34 domain, or is an amino acid sequence having at least about 80%, 85%, 90%, 95%, 98% or 99% sequence identity to the CD3 domain.

Polynucleotides, mRNA, and Methods of Expressing or Preparing Antibodies

The present disclosure also provides polynucleotides or nucleic acid molecules encoding the antibodies, variants or derivatives thereof of the disclosure, or the CAR. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

In some embodiments, the polynucleotide is an mRNA molecule. In some embodiments, the mRNA can be introduced into a target cell for expressing the antibody or fragment thereof.

mRNAs may be synthesized according to any of a variety of known methods. For example, the mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, for the preparation of antibody-coding mRNA, a DNA template is transcribed in vitro. A suitable DNA template typically has a promoter, for example a T3, T7 or SP6 promoter, for in vitro transcription, followed by desired nucleotide sequence for desired antibody encoding (e.g., heavy chain or light chain encoding) mRNA and a termination signal.

Desired antibody encoding (e.g., heavy chain or light chain encoding) mRNA sequence may be determined and incorporated into a DNA template using standard methods. For example, starting from a desired amino acid sequence (e.g., a desired heavy chain or light chain sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

The mRNA may be synthesized as unmodified or modified mRNA. Typically, mRNAs are modified to enhance stability. Modifications of mRNA can include, for example, modifications of the nucleotides of the RNA. A modified mRNA can thus include, for example, backbone modifications, sugar modifications or base modifications. In some embodiments, antibody encoding mRNAs (e.g., heavy chain and light chain encoding mRNAs) may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 13-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosure of which is included here in its full scope by reference.

In some embodiments, the mRNAs (e.g., heavy chain and light chain encoding mRNAs) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, the mRNAs (e.g., heavy chain and light chain encoding mRNAs) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, the mRNAs (e.g., heavy chain and light chain encoding mRNAs) may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, the mRNAs (e.g., heavy chain and light chain encoding mRNAs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5)A and G(5)ppp(5′)G.

In some embodiments, the mRNAs (e.g., heavy chain and light chain encoding mRNAs) include a 3′ poly(A) tail structure. A poly-A tail on the 3′ terminus of mRNA typically includes about 10 to 300 adenosine nucleotides (e.g., about 10 to 200 adenosine nucleotides, about 10 to 175 adenosine nucleotides, about 10 to 150 adenosine nucleotides, about 10 to 125 adenosine nucleotides, 10 to 100 adenosine nucleotides, about 10 to 75 adenosine nucleotides, about 20 to 70 adenosine nucleotides, or about 20 to 60 adenosine nucleotides). In some embodiments, antibody encoding mRNAs (e.g., heavy chain and light chain encoding mRNAs) include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the mRNAs (e.g., heavy chain and light chain encoding mRNAs) include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length (e.g., about 50 and 400 nucleotides in length, about 50 and 300 nucleotides in length, about 50 and 200 nucleotides in length, or about 50 and 100 nucleotides in length).

In some embodiments, a 5′ region of an mRNA (e.g., heavy chain and light chain encoding mRNAs) includes a sequence encoding a signal peptide, such as those described herein. In particular embodiments, a signal peptide derived from human growth hormone (hGH) is incorporated in the 5′ region. Typically, a signal peptide encoding sequence is linked, directly or indirectly, to the heavy chain or light chain encoding sequence at the N-terminus.

The present technology may be used to deliver any antibody known in the art and antibodies that can be produced against desired antigens using standard methods. The present invention may be used to deliver monoclonal antibodies, polyclonal antibodies, antibody mixtures or cocktails, human or humanized antibodies, chimeric antibodies, or bi-specific antibodies.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment and Uses

As described herein, the antibodies, variants, antibody-drug conjugates, chimeric antigen receptors (CAR) and CAR cells, encoding polynucleotides or derivatives of the present disclosure may be used in certain treatment and diagnostic methods.

The present disclosure is further directed to antibody-based therapies which involve administering the antibodies or fragments, antibody-drug conjugates, chimeric antigen receptors (CAR) and CAR cells, encoding polynucleotides or derivatives of the disclosure to a patient such as an animal, a mammal, and a human for treating one or more of the disorders or conditions described herein. Therapeutic molecules or cells of the disclosure include, but are not limited to, antibodies of the disclosure (including variants and derivatives thereof as described herein), antibody-drug conjugates, chimeric antigen receptors (CAR) and CAR cells, and nucleic acids or polynucleotides encoding antibodies of the disclosure (including variants and derivatives thereof as described herein).

The molecules or cells of the disclosure can also be used to treat or inhibit cancer. As provided above, B7-H3 can be overexpressed in tumor cells, in particular liver, gastric, pancreatic, esophageal, ovarian, and lung tumors. Inhibition of B7-H3 has been shown to be useful for treating the tumors.

Accordingly, in some embodiments, provided are methods for treating a cancer in a patient in need thereof. The method, in one embodiment, entails administering to the patient an effective amount of the molecules or cells of the present disclosure. In some embodiments, at least one of the cancer cells (e.g., stromal cells) in the patient over-express B7-H3.

Cellular therapies, such as chimeric antigen receptor (CAR) T-cell therapies, are also provided in the present disclosure. A suitable cell can be used, that is transduced with a vector that encodes, or put in contact with, an CAR that includes an anti-B7-H3 antibody of the present disclosure (or alternatively engineered to express an anti-B7-H3 antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma. A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibody or fragment include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracisternally, intravaginally, intraperitoneally, topically (as by powders, ointments, drops or transdermal patch), buccally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies or fragments of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antibodies or fragments of the present disclosure is typically 0.001 mg/kg to 100 mg/kg of the patient's body weight, between 0.01 mg/kg and 20 mg/kg of the patient's body weight, or 0.5 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody or fragment, antibody-drug conjugates, chimeric antigen receptors (CAR) and CAR cells, encoding polynucleotides, or derivatives and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1. Generation and Testing of Mouse Anti-Human B7-H3 Antibodies

This example describes the generation of mouse anti-human B7-H3 monoclonal antibodies using the hybridoma technology.

Antigen: Human B7-H3(41g) His tag protein. Protein immunization was performed by subcutaneous injection (s.c.) and intraperitoneal injection (i.p.).

Immunization: To generate monoclonal antibodies against human B7-H3, Balb/c mice, C57BL/6 mice, SJL mice, and A/J mice, were immunized with the human B7-H3(4Ig) protein. To monitor immune responses, titrated serum from mice was screened by ELISA and flow cytometry as described below. Serum was screened by antibody binding to human B7-H3 protein and B7-H3 overexpressed CHO-K1 cell line, and the negative control CHO-K1 cell line not expressing B7-H3. Animals with enough ELISA titers and FACS binding ratio of positive cell line (B7-H3 overexpressed CHO-K1 cell) over negative cell line (CHO-K1 expressing vector only) were selected for final boost with B7-H3 protein before hybridoma fusions.

Cell fusion and Hybridoma screening: The spleens were isolated from final boosted mice as described above. Hybridomas were generated by cell fusions with immortalized mouse myeloma cells by electric field-based electrofusion. The fused cells were plated in 96 flat-bottom microtiter plates for selection of hybridomas. The supernatants were screened by epitope binning using ELISA and FACS using CHO-K1 overexpressing B7-H3 as well as the negative control cell line.

Subcloning and screening: Positive primary clones with different epitopes from each fusion were subcloned by limiting dilutions to ensure that the subclones were derived from a single parental cell. Subcloning was screened in the same approach as primary clones and additionally, to confirm the binding potency of antibodies, culture supernatants of positive clones were tested by FACS on A375 cells.

Hybridoma clones were selected for further analysis. The amino acid sequences of the variable regions are provided in Table 1A below.

TABLE 1A
Antibody Variable Region Sequences

| | Sequence | SEQ ID |
|---|---|---|
| Name | (CDRs according to Kabat system are underlined) | NO: |
| 35A12B11 VH | QVQLQQPGAELVKPGASVKLSCKASGYTFTSYWMHWVKQRPGQGLEWIGM | 1 |
| | IHPNSGTTNYNEKFKNKATLTVDKSSSTAYMQLSSLTSEDSAVYYFTRNGNP | |
| | RWCFDVWGTGTTVTVSS | |
| 35A12B11 VL | ENVLTQSPAIMSASPGEKVTVTCSASSSVSYMHWYQQKSSTSPKLWIYDTSKL | 2 |
| | ASGVPGRFSGSGSGNSYSLTISSMEAEDVATYYCFQGSGYPFTFGSGTKLEIK | |
| 62H9H5 VH | QVQLQQSGAELAKPGASVKMSCKASGYTFTNYWIHWVKQRPGQGLEWIGYI | 3 |
| | SPSTGYTEYNQKFKGKATLTAVKSSSTAFMQLSSLTSEDSAVYFCARGTGYW | |
| | GQGTTLTVSS | |
| 62H9H5 VL | DIQMTQSPASLSASVGESVTITCRASENIYSFFAWYQQKEGKSPQLLVYNAKT | 4 |
| | LEEGVPSRFSGSGSGTQFSLKINSLQPEDFGSYYCQHNYVSPFTFGSGTKLEIK | |
| 72D1D11 VH | QIQLVQSGPELKKPGETVKISCKASGYTFTNLGLNWVKQAPGKGLKWMGWI | 5 |
| | NTAGGQPTYAEDFKGRCAFSLETSASTAYLQINNLKNEDTATYFCARDDGYY | |
| | YTMDYWGQGTSVTVSS | |
| 72D1D11 VL | DIVMTQTPLSLPVSLGDQASISCRSSQSLVHSNGKTYLHWYLQKPGQSPKLLI | 6 |
| | YKVSNRFSGVPDRFSGSGSGTDFTLKISRVEAEDLGVYFCSQSTHVPWTFGG | |
| | GTKLEIK | |
| 81C8A1 VH | QVTLKESGPGILQPSQTLSLTCSFSGFSLSTSGMGVSWIRQPPGKGLEWLAHIY | 7 |
| | WDDDRRYNPSLKSRLTISRDTSRNQVFLQITTVDTADTATYYCARSPYGSEAY | |
| | WGQGTLVIVSA | |
| 81C8A1 VL | MQIVLSQSPTILSAFPGEKVTMTCRASSSVSFIQWYQQKSGSSPKPWIYSPSNL | 8 |
| | ASGVPTRFSGSGSGTSYSLTIGRVEAEDAATYYCQQWSSYPWTFGGGTKVEIK | |
| 97E6B2 VH | QVQLQQPGAELVKPGASVKMSCKASGYTFTSYWITWVKQRPGQGLEWIGDI | 9 |
| | YPGSNNTYYNEKFKSKATLTVDTSSSTAYIQLSSLTSEDSAVYYCARLIMSLF | |
| | DYSGQGTTLTVSS | |
| 97E6B2 VL | ENVLTQSPANMSASPGEKVTMTCSVSSSVSYIHWYQQKSSTSPTLWIYDTSKL | 10 |
| | ASGVPGRFSGSGSGNSYALTISSMEAEDVATYYCFQGSAYPLTFGAGTKLELK | |
| 106A5B3 VH | EVQLVESGGDLVKPGGSLKLSCAASGFTFSSYGMSWVRQTPDKRLEWVATIS | 11 |
| | SGGSYIYYPDSMKGRFTISRDNAKNTLYLQMSSLQSEDTAIYYCASDYGSRY | |
| | FDYWGQGTTLTVSS | |
| 106A5B3 VL | QIVLTQSPAIMSASPGEKVTMTCSASSSVIYMSWYKQKPGSSPRLLIYDTSNLA | 12 |
| | SGVPVRFSGSGSGTSYSLTISRMEAEDAATYYCQQWSSYPRTFGGGTKLEIK | |
| 126C10B10 VH | GVQLQQSGAELVKPGASVKLSCTGSGFNIKDPYIHWVKQRPEQGLEWIGRID | 13 |
| | PANGNTKYGPRFQGRATMTADTSSNTANLQLSSLTSEDTAVYYCTTWNYYL | |
| | SRGFDYWGQGTTLTVSS | |
| 126C10B10 VL | QIVLTQSPAIMSASPGEKVTMTCSARSSVTYMHWYQQKSGTSPKRWIYDTSK | 14 |
| | LASGVPARFSGSGSGTSYSLTISSMEAEDAATYYCQQWTSNPLTFGAGTKLEL | |
| | K | |
| 216E7A9 VH | DVQLVESGGGLVQPGGSRKLSCAASGFTFSSFGMHWVRQAPEKGLEWVAYIS | 15 |
| | SGSSTIYYADTVRGRFTISRDNPKNTLFLQMTSLRSEDTAMYYCARGGDYYY | |
| | ALDYWGQGTSVTVSS | |
| 216E7A9 VL | DIQMTQSPASLSVSVGETVTITCRASENIHSNLEWYQQKQGKSPQLLVYAATN | 16 |
| | LADGVPSRFSGSGSGTQYSLKINSLQSEDFGSYYCQHFWGTPFTFGSGTKLEI | |
| | K | |
| 227E12D1 VH | EVKLVESGGGLVKPGGSLKLSCAASGFTFSSYGMSWVRQTPEKRLEWVATIS | 17 |
| | GGGTYTYYPDSVKGRFTISRDNAKNNLYLQMSSLRSDDTALYYCARHEGLV | |
| | MDYWGQGTSVTVSS | |
| 227E12D1 VL | DIQMTQSPASLSASVGETVTITCRASENIYSYLAWYQQKQGKSPQLVVYNAK | 18 |
| | TLAEGVPSRFSGSGSGTQFSLKINSLQPEDFGTYYCQHHYGPPPYTFGGGTKL | |
| | EIK | |
| 294A3C4 VH | QVQLQQPGAELVKPGASVKLSCKASGYTFTSYWMHWVKQRPGQGLEWIGM | 19 |
| | FHPNSGTTNYNEKFKSKATLTVDKSSSTAYMQLSSLTSEDSAVYYCARRLGIF | |
| | FDYWGQGTTLTVSS | |
| 294A3C4 VL | DIVMTQSPSSLTVTAGEKVTMSCRSSQSLLNSGNQKNYLTWYQQKPGQPPKL | 20 |
| | LIYWASTRDSGVPDRFTGSGSGTDFTLTISSVQTEDLAVYYCQTDYSYPFTFG | |
| | SGTKLEIK | |
| 312E1E2 VH | QVQLQQSGPELVRPGVSVKISCKGSGYTFTDYAMHWVKQSHAKSLEWIGIVS | 21 |
| | THYGNTNYNQNFKGKATMTVDKSSSTVYMELARLTSEDSAIYYCARSEGLR | |
| | LDWFAHWGQGTLVTVSA | |
| 312E1E2 VL | DIQMTQSPASLSASVGEIVTITCRASENIYSYLAWYQQKQGKSPHLLVYNAKT | 22 |
| | LAEGVPSRFSGSGSGTQFSLKISRLQPEDFGSYYCQNHYVFPFTFGSGTKLEIK | |
| 429H9F10 VH | QIQLVQSGPELKKPGETVKISCKASGYTFTYYGLNWVKQAPGKGLRWMGWI | 23 |
| | NTSSGVPTYADDFKGRFAISLESSASTVFLEISNLKNEDTAAYFCAKDDGYFY | |
| | TMDHWGQGTSVTVSS | |
| 429H9F10 VL | DIVMTQPPLSLPVSLGDQASISCRSSQSLVHNNGKTYLHWYLQKPGQSPKLLI | 24 |
| | YKVSNRFSGVPDRFSGSGSGTDFTLKISRVETEDLGVYFCSQSTQVPWTFGG | |
| | GTKLEIK | |
| 43G8A9 VH | QVQLQQPGAELVKPGASVKLSCKASGYTFTSYWMHWVNQRPGQGLEWIGM | 180 |
| | IHPNSGSTNYNEKFKSKATLTVDKSSSTAYMQLSSLTSEDSAVYYCARNYGR | |
| | YFDYWGQGTTLTVSS | |
| 43G8A9 VL | MDFQVQIFSFLLISVSVIMSRGQIVLTQSPAIMSASPGEKVTLTCSANSSVSSSSL | 181 |
| | YWYQQKPGSSPKLWIYSTSNLASGVPARFSGSGSGTSYSLTISSMEAEDAASY | |
| | FCHQWSSYPLTFGAGTKLELK | |

The CDR sequences of these antibodies (according to Kabat system) are listed in Table 1B below. Also shown are their variants in which potential sites for post-translational modification (PTM) are mutated to biological equivalents (e.g., NG=>NA, NG=>QG, NS=>NA, NS=>QS, NS=>YS, DG=>DA, DG=>EG and DS=>DA). These substitutions are contemplated to retain the biological activities of the antibodies while preventing PTM, to facilitate manufacturing.

TABLE 1B
CDR Sequences (with optional mutations to avoid PTM, Kabat numbering)

| | | SEQ ID | | SEQ | | SEQ |
|---|---|---|---|---|---|---|
| Antibody | VH CDR1 | NO: | VH CDR2 | ID NO: | VH CDR3 | ID NO: |
| 35A12B11 | SYWMH | 25 | MIHPNSGTTNYNEKFKN | 26 | NGNPRWCFDV | 29 |
| | | | MIHPQSGTTNYNEKFK | 27 | | |
| | | | N | 28 | | |
| | | | MIHPYSGTTNYNEKFKN | | | |
| 62H9H5 | NYWIH | 33 | YISPSTGYTEYNQKFKG | 34 | GTGY | 35 |
| 72D1D11 | NLGLN | 39 | WINTAGGQPTYAEDFK | 40 | DDGYYYTMDY | 41 |
| | | | G | | DDAYYYTMDY | 42 |
| | | | | | DEGYYYTMDY | 43 |
| | | | | | DRGYYYTMDY | 44 |
| | | | | | DLGYYYTMDY | 45 |
| | | | | | DAGYYYTMDY | 46 |
| 81C8A1 | TSGMGV | 52 | HIYWDDDRRYNPSLKS | 53 | SPYGSEAY | 54 |
| | S | | | | | |
| 97E6B2 | SYWIT | 58 | DIYPGSNNTYYNEKFKS | 59 | LIMSLFDY | 60 |
| 106A5B3 | SYGMS | 64 | TISSGGSYIYYPDSMKG | 65 | DYGSRYFDY | 67 |
| | | | TISSGGSYIYYPDAMKG | 66 | | |
| 126C10B10 | DPYIH | 71 | RIDPANGNTKYGPRFQG | 72 | WNYYLSRGFD | 75 |
| | | | RIDPAQGNTKYGPRFQG | 73 | Y | |
| | | | RIDPANANTKYGPRFQG | 74 | | |
| 216E7A9 | SFGMH | 79 | YISSGSSTIYYADTVRG | 80 | GGDYYYALDY | 81 |
| 227E12D1 | SYGMS | 85 | TISGGGTYTYYPDSVKG | 86 | HEGLVMDY | 88 |
| | | | TISGGGTYTYYPDAVKG | 87 | | |
| 294A3C4 | SYWMH | 92 | MFHPNSGTTNYNEKFK | 93 | RLGIFFDY | 97 |
| | | | S | 94 | | |
| | | | MFHPNAGTTNYNEKFK | 95 | | |
| | | | S | 96 | | |
| | | | MFHPQSGTTNYNEKFK | | | |
| | | | S | | | |
| | | | MFHPYSGTTNYNEKFK | | | |
| | | | S | | | |
| 312E1E2 | DYAMH | 102 | IVSTHYGNTNYNQNFK | 103 | SEGLRLDWFAH | 104 |
| | | | G | | | |
| 429H9F10 | YYGLN | 108 | WINTSSGVPTYADDFKG | 109 | DDGYFYTMDH | 110 |
| | | | | | DDAYFYTMDH | 111 |
| | | | | | DEGYFYTMDH | 112 |
| 43G8A9 | SYWMH | 182 | MIHPNSGSTNYNEKFKS | 183 | NYGRYFDY | 187 |
| | | | MIHPNAGSTNYNEKFKS | 184 | | |
| | | | MIHPQSGSTNYNEKFKS | 185 | | |
| | | | MIHPYSGSTNYNEKFKS | 186 | | |
| | | SEQ | | SEQ ID | | SEQ ID |
| Antibody | VL CDR1 | ID NO: | VL CDR2 | NO: | VL CDR3 | NO: |
| 35A12B11 | SASSSVSYMH | 30 | DTSKLAS | 31 | FQGSGYPFT | 32 |
| 62H9H5 | RASENIYSFFA | 36 | NAKTLEE | 37 | QHNYVSPFT | 38 |
| 72D1D11 | RSSQSLVHSNGKTYLH | 47 | KVSNRFS | 50 | SQSTHVPWT | 51 |
| | RSSQSLVHSNAKTYLH | 48 | | | | |
| | RSSQSLVHSQGKTYLH | 49 | | | | |
| 81C8A1 | RASSSVSFIQ | 55 | SPSNLAS | 56 | QQWSSYPWT | 57 |
| 97E6B2 | SVSSSVSYIH | 61 | DTSKLAS | 62 | FQGSAYPLT | 63 |
| 106A5B3 | SASSSVIYMS | 68 | DTSNLAS | 69 | QQWSSYPRT | 70 |
| 126C10B10 | SARSSVTYMH | 76 | DTSKLAS | 77 | QQWTSNPLT | 78 |
| 216E7A9 | RASENIHSNLE | 82 | AATNLAD | 83 | QHFWGTPFT | 84 |
| 227E12D1 | RASENIYSYLA | 89 | NAKTLAE | 90 | QHHYGPPPYT | 91 |
| 294A3C4 | RSSQSLLNSGNQKNYLT | 98 | WASTRDS | 99 | QTDYSYPFT | 101 |
| | | | WASTRDA | 100 | | |
| 312E1E2 | RASENIYSYLA | 105 | NAKTLAE | 106 | QNHYVFPFT | 107 |
| 429H9F10 | RSSQSLVHNNGKTYLH | 113 | KVSNRFS | 116 | SQSTQVPWT | 117 |
| | RSSQSLVHNNAKTYLH | 114 | | | | |
| | RSSQSLVHNQGKTYLH | 115 | | | | |
| 43G8A9 | SANSSVSSSSLY | 188 | STSNLAS | 192 | HQWSSYPLT | 193 |
| | SANASVSSSSLY | 189 | | | | |
| | SAQSSVSSSSLY | 190 | | | | |
| | SAYSSVSSSSLY | 191 | | | | |

Bold/underlined residues indicate mutations to avoid PTM

### Example 2: Full Human Naïve Phage Library Panning and Screening

This example screened for full human anti-B7-H3 antibodies from a human Naïve phage library.

Antigen: human B7-H3 (2Ig)-Avi-biotin, human B7-H3 (4Ig) His tag, human B7-H3 (4Ig)-Avi-biotin-His.

Preparation of full human naïve phage library: the phage library was constructed by using phagemid vectors which consisted of antibody gene fragments that were amplified from PBMCs of healthy human subjects. The library format was Fab phage library. The library size was 1.04×10¹¹.

Solid phase and solution panning of phage libraries against human B7-H3 protein: For solution panning, the phage libraries were first performed negative screening by incubating with BSA-coated streptavidin Dynabeads. The resulting phages were incubated with Biotinylated-B7-H3-His tag protein and washed by Kingfisher magnetic beads system. The binders were eluted by trypsin. For solid phase panning, the phage libraries were firstly blocked by PBS supplemented with 5% milk. The resulting phages were incubated with B7-H3 His tag protein and washed by PBST. The binders were eluted by trypsin.

88e The eluted phages were subsequently tested for their titers to bind antigens and co-cultured with E. coli. There were four rounds of panning and screening. The titers of output 3 and output 4 were significantly increased.

Single clones were cherrypicked from output 3 and 4 and then cultured in 96 well deep plates. The culture supernatant was subject to IgG concentration and antigen binding titer evaluation. A total of 232 positive clones were selected and subject to sequencing. Post-sequence analysis identified 65 unique sequences. All these clones were subjected to ELISA binding analysis and sequences of strong binders were showed in Table 2A below.

TABLE 2A
Antibody Variable Region Sequences

| | Sequence | SEQ ID |
|---|---|---|
| Clone | (CDRs according to Kabat system are underlined) | NO: |
| AHP05564 VH | EVQLVETGGGVVQPGRSLRLSCAASGFTFSSSGMHWVRQAPGKGL | 118 |
| | EWVAVISYDVTNKYYVDSVKGRFTISRDNSENTLYLQMNSLRAED | |
| | TGVYYCVRDGGPRWLGWYFDLWGRGTLVTVSS | |
| AHP05564 VL | NIQMTQSPSSQSASVGDRVTITCRANQAIANSLAWYQQKPGKAPKL | 119 |
| | LLFGTSLLHSGVPSRFSGSGSGTEYTLTISSLQPDDFGTYYCQQYFS | |
| | FPRTFGQGTQVDVY | |
| AHP06331 VH | EVQLVQSGGGLVQPGGSLRLSCAASGFTFSSYAMSWVRQAPGKGL | 120 |
| | EWVSAISGSGGSTYYADSVKGRFTISRDNSKNTLYLQMNSLRAEDT | |
| | AVYYCAKDRYYDILTGTFGYWGQGTLVTVSS | |
| AHP06331 VL | EIVMTQSPDFQSVTPKEKVTITCRASQSIGSSLHWYQQKPDQSPKLL | 121 |
| | IKHASQPISGVPSRFSGSGSGTVFTLTIQSLEAEDAATYYCQQSSRLP | |
| | TFGGGTKVEIQ | |
| AHP06345 VH | QVQLVQSGAEVKKPGESLRISCKGSGYSFTSYWISWVRQMPGKGLE | 122 |
| | WMGRIDPSDSYTNYSPSFQGHVTISADKSISTAYLQWSSLKASDTA | |
| | MYYCARHTGYYGSGIGMDVWGQGTLVTVSS | |
| AHP06345 VL | HVILTQPPSVSAAPGQKVTISCSGSSSNIGNNYVSWYRQLPGTAPKL | 123 |
| | LIYDNNQRPSGIPDRFSGSKSGTSATLGITGLQTGDEADYYCATWDS | |
| | SLGVVVFGGRTKLTVL | |
| AHP06358 VH | EVQLVQSGAEVKKPGASVKVSCKASGYTFTSYGISWVRQAPGQGL | 124 |
| | EWMGWISAYNGNTNYAQKLQGRVTMTTDTSTSTAYMELRSLRSD | |
| | DTAVYYCARDSPLQIAVAGYYYGMDVWGQGTTVTVSS | |
| AHP06358 VL | NFMLTQPHSVSESPGKTVTISCTRSSGSIVSGYVQWYQQRPGSAPST | 125 |
| | VIYEHNQRPSGVPDRFSGSIDSSSNSASLTISGLRTEDEADYYCQSY | |
| | DSRNMIFGGGTKLTVL | |

The CDR sequences of these antibodies (according to Kabat system) are listed in Table 2B below. Also shown are their variants in which potential sites for post-translational modification (PTM) are mutated to biological equivalents (e.g., NG=>NA, NG=>QG, NS=>NA, NS=>QS, NS=>YS, DG=>DA, DG=>EG and DS=>DA). These substitutions are contemplated to retain the biological activities of the antibodies while preventing PTM, to facilitate manufacturing.

TABLE 2B
CDR Sequences (with optional mutations to avoid PTM, Kabat numbering)

| | | SEQ | | SEQ | | SEQ |
|---|---|---|---|---|---|---|
| | | ID | | ID | | ID |
| Antibody | VH CDR1 | NO: | VH CDR2 | NO: | VH CDR3 | NO: |
| AHP05564 | SSGM | 126 | VISYDVTNKYYVDSV | 127 | DGGPRWLGWYFDL | 129 |
| | H | | KG | | DAGPRWLGWYFDL | |
| | | | VISYDVTNKYYVDAV | 128 | EGGPRWLGWYFDL | 130 |
| | | | KG | | | 131 |
| AHP06331 | SYAM | 135 | AISGSGGSTYYADSV | 136 | DRYYDILTGTFGY | 138 |
| | S | | KG | | | |
| | | | AISGSGGSTYYADAV | 137 | | |
| | | | KG | | | |
| AHP06345 | SYWIS | 142 | RIDPSDSYTNYSPSFQ | 143 | HTGYYGSGIGMDV | 144 |
| | | | G | | | |
| AHP06358 | SYGIS | 149 | WISAYNGNTNYAQK | 150 | DSPLQIAVAGYYYG | 153 |
| | | | LQG | 151 | MDV | |
| | | | WISAYQGNTNYAQK | | | |
| | | | LQG | 152 | DAPLQIAVAGYYYG | 154 |
| | | | WISAYNANTNYAQK | | MDV | |
| | | | LQG | | | |
| | | SEQ | | SEQ | | SEQ |
| | | ID | | ID | | ID |
| Antibody | VL CDR1 | NO: | VL CDR2 | NO: | VL CDR3 | NO: |
| AHP05564 | RANQAIANSLA | 132 | GTSLLHS | 133 | QQYFSFPRT | 134 |
| AHP06331 | RASQSIGSSLH | 139 | HASQPIS | 140 | QQSSRLPT | 141 |
| AHP06345 | SGSSSNIGNNYVS | 145 | DNNQRPS | 146 | ATWDSSLGVVV | 147 |
| | | | | | ATWDASLGVVV | 148 |
| AHP06358 | TRSSGSIVSGYVQ | 155 | EHNQRPS | 156 | QSYDSRNMI | 157 |
| | | | | | QSYDARNMI | 158 |

Bold/underlined residues indicate mutations to avoid PTM

### Example 3. Binding Activity to Human B7-H3 Antigen

This example tested the binding activities of the antibodies to the human B7-H3 antigen.

### 3.1 Cell Based Binding to Human B7-H3

To evaluate the binding activity of clones, the tested antibodies were subjected to FACS test on A375 or A375.S2 cells.

Briefly, a total number of 4×10⁴ A375 cells was first incubated with 3-fold or 5-fold serially diluted antibodies at 4° C. for 30 mins in FACS buffer. After washing by FACS buffer, fluorescent dye conjugated-anti-human IgG antibody was added to each well and incubated at 4° C. for 30 mins. Samples were washed twice with FACS buffer. The geometric mean florescence intensity (MFI) was evaluated by MACSQuant Analyzer 16.

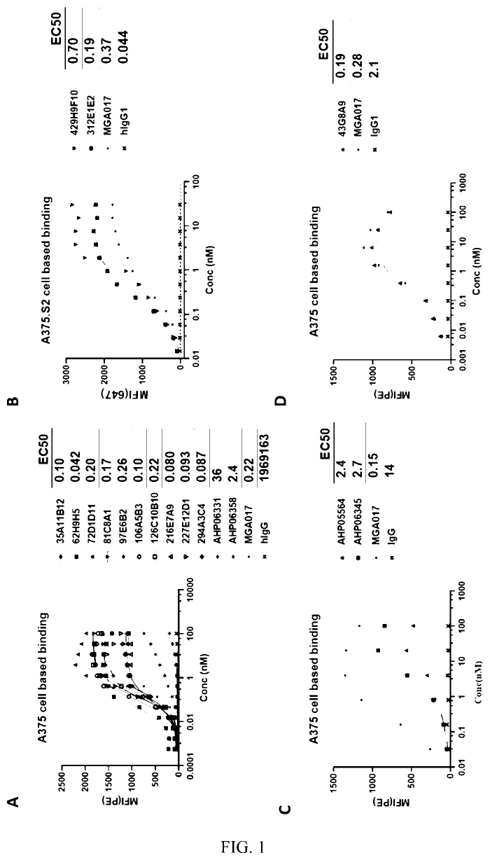

As shown in FIG. 1, among all these hybridoma clones, 43G8A9 showed comparable binding to MGA017, others showed better binding activity than MGA017, a positive reference. Four antibodies derived from human naïve phage library showed weaker binding activity than MGA017.

### 3.2 the Kinetic Activity to Human B7-H3-4Ig

The binding of the tested antibodies to human B7-H3 (4Ig) protein (his tag) was tested with Biacore using a capture method. The mAbs were captured by Protein A chip. A serial dilution of human B7-H3 protein was injected over captured antibody for 3 mins at a flow rate of 30 μL/min. The antigen was allowed to dissociate for 600s. All the experiments were carried out on a Biacore T200. Binding traces were fitted by the Biacore T200 evaluation software according to 1:1 model to calculate the association (ka) and dissociation (kd) rate constants as well as the equilibrium constant (KD). The results are shown in Table 3 below.

TABLE 3
Kinetic results measured by Biacore

Human B7H3α-His

| Abs | ka (1/Ms) | kd (1/s) | KD (M) |
|---|---|---|---|
| MGA017 | 7.37 × 10⁵ | 1.32 × 10⁻³ | 1.79 × 10⁻⁹ |
| 35A12B11 | 5.29 × 10⁵ | 2.01 × 10⁻³ | 3.81 × 10⁻⁹ |
| 62H9H5 | 3.46 × 10⁵ | 1.60 × 10⁻⁴ | 4.64 × 10⁻¹⁰ |
| 72D1D11 | 3.74 × 10⁵ | 1.80 × 10⁻⁴ | 4.82 × 10⁻¹⁰ |
| 81C8A1 | 5.67 × 10⁵ | 1.11 × 10⁻⁴ | 1.96 × 10⁻¹⁰ |
| 97E6B2 | 6.47 × 10⁵ | 4.81 × 10⁻⁴ | 7.43 × 10⁻¹⁰ |
| 106A5B3 | 8.33 × 10⁵ | 3.96 × 10⁻⁴ | 4.76 × 10⁻¹⁰ |
| 126C10B10 | 4.38 × 10⁵ | 3.67 × 10⁻⁴ | 8.37 × 10⁻¹⁰ |
| 216E7A9 | 3.31 × 10⁵ | 8.24 × 10⁻⁴ | 2.49 × 10⁻⁹ |
| 227E12D1 | 5.95 × 10⁵ | 3.68 × 10⁻⁴ | 6.19 × 10⁻¹⁰ |
| 294A3C4 | 4.63 × 10⁵ | 1.35 × 10⁻³ | 2.92 × 10⁻⁹ |
| AHP05564 | 1.66 × 10⁶ | 8.62 × 10⁻⁴ | 5.20 × 10⁻¹⁰ |
| AHP06331 | 9.06 × 10⁴ | 3.72 × 10⁻³ | 4.11 × 10⁻⁸ |
| AHP06345 | 1.73 × 10⁵ | 2.54 × 10⁻⁴ | 1.47 × 10⁻⁹ |
| AHP06358 | 5.37 × 10⁶ | 3.96 × 10⁻³ | 7.38 × 10⁻¹⁰ |

### 3.3 the Binding Epitope of Tested Antibodies

The binding epitope of tested antibodies was characterized by epitope binning assay using Octet.

Briefly, the human B7-H3 protein at a concentration of 100 nM was captured on the sensor, leading to a signal of 0.3 nm. Then, the sensors were transferred into the 100 nM competing antibodies (72D1D11, AHP05562, AHP06345, CA84D) were injected into the sensor for antigen saturation and association for 120s. Then, tested antibodies were injected for antigen competition testing for another 120s. The sensors were transferred into the 100 nM tested antibodies for 120s after a baseline step. Data analysis was carried out using the Octet Analysis Studio 12.2 software. The binding inhibition ratio between tested antibodies and competing antibodies are shown in Table 4A below. Based on the results, the binding epitope of tested antibodies can be classified as Bin Aα, Aβ, Aγ, B and C, as shown in Table 4B.

TABLE 4A
Inhibition ratio tested by Octet

| | Competitive Ab | | | |
|---|---|---|---|---|
| Tested Ab | 72D1D11 | AHP05562 | AHP06345 | CA84D |
| MGA017 | 98.91% | 91.49% | −0.09% | 3.65% |
| 35A12B11 | 3.44% | 92.01% | −7.09% | −9.56% |
| 62H9H5 | 95.55% | 90.21% | −7.10% | −6.07% |
| 72D1D11 | 96.14% | −9.17% | −5.22% | −5.46% |
| 81C8A1 | 95.40% | 90.64% | −12.05% | −6.30% |
| 97E6B2 | 99.65% | −7.80% | −1.18% | −3.99% |
| 106A5B3 | 96.77% | 89.58% | −13.04% | −15.75% |
| 126C10B10 | 96.60% | −22.23% | −5.98% | −9.58% |
| 216E7A9 | 98.03% | 92.30% | 1.99% | 5.28% |
| 227E12D1 | 97.11% | 89.98% | −5.22% | −5.46% |
| 294A3C4 | −5.00% | 89.96% | −7.53% | −12.01% |
| 312E1E12 | −32.57% | −24.39% | 89.96% | −18.26% |
| 429H9F10 | 98.86% | −22.36% | −11.54% | −8.00% |
| 43G8A9 | −0.01% | 91.22% | −0.02% | −0.02% |
| AHP05564 | Not tested | −3.53% | 5.92% | 80.14% |
| AHP06331 | −8.37% | −11.83% | −13.00% | 82.66% |
| AHP06345 | Not tested | −6.36% | 91.81% | −3.77% |
| AHP06358 | −13.90% | 91.72% | −10.55% | −13.62% |

TABLE 4B
Epitope binning results

| Bin | Antibody |
|---|---|
| Aα | MGA017, 62H9H5, 81C8A1, 106A5B3, 216E7A9, 227E12D1 |
| Aβ | 72D1D11, 97E6B2, 126C10B10, 429H9F10 |
| Aγ | 35A12B11, 294A3C4, AHP06358, 43G8A9 |
| B | 312E1E12, AHP06345 |
| C | AHP06331, AHP05564 |

### Example 4. Internalization of Tested Antibodies

This example characterized the internalization of tested antibodies in A375 cells.

pHAb thiol Dyes are pH-sensitive dyes that have very low fluorescence at pH>7, and a dramatic increase in fluorescence will occur when it is internalized into the endosome or lysosome where the pH is around 6.3 or 4.7, respectively. Briefly, a pHAb thiol Dyes labeled a-hIgG secondary antibody (10 nM) was incubated with serially diluted antibodies starting from 5-50 nM for 30 min. And the mixture was added into 96-well assay plates pre-seeded with 2×10⁴ A375 cells in each well. After 48 hours of culture, fluorescence signal was captured on Multimode Plate Reader (Envision® 2105) or Operetta.

As shown in FIG. 2, almost all hybridoma clones showed better internalization than MGA017. Among the antibodies derived from human naïve phage library, AHP06331 and AHP06358 showed less extent of internalization than MGA017 at lower concentrations, but at higher concentrations, the internalization of AHP06331 and AHP06358 were much stronger than MGA017, suggested by higher EC50 and higher top value of the assay. AHP05564 and AHP05346 showed weaker internalization than MGA017.

### Example 5. In Vitro Cytotoxicity of B7-H3 ADCs

This example characterized the killing efficacy of vc-MMAE labeled B7-H3 ADCs on RKO tumor cells.

B7-H3 leading hits were labeled with vc-MMAE(L1-D1), with a drug-to-antibody ratio (DAR) of ˜-4.0. The cytotoxicity of B7-H3 ADCs was tested on RKO cells. Briefly, a number of 2×10⁴ RKO cells were seeded in each well of 96 well plates overnight. Serially diluted B7-H3 ADCs were added to the cell culture and the mixture was incubated in a CO₂ incubator at 37° C. for 72 hours or 96 hours. At the end of the incubation, 100 μL CellTiter-Glo reaction reagent was added and the mixture was incubated at room temperature for 10 minutes. The luminescence signal was obtained by Multimode Plate Reader (Envision® 2105).

As shown in FIG. 3, 72D1D11, 81C8A1, 62H9H5, 106A5B3, 126C10B10, and 227E12D1 mediated stronger cytotoxicity against RKO cell killing, compared to MGA017. AHP06358 showed comparable cell killing to MGA017.

### Example 6. Humanization of the B7-H3 Antibodies

The variable region genes of antibodies 72D1D11 and 81C8A1 were employed to generate humanized mAb. The amino acid sequences of the VH and VK of 72D1D11 and 81C8A1 were compared against the available database of human Ig gene sequences to find the overall best-matching human germline Ig sequences.

For the light chain of 72D1D11, IGKV2-40*01 and IGKV2-28*01 is the best fit germline, and for the heavy chain of 72D1D11, IGHV7-4-1*02 was chosen as the humanization backbone. Humanized 72D1D11 CDR grafting antibody was then designed where the CDR-L1, L2 and L3 were grafted onto framework sequences of the IGKV2-40*01 and IGKV2-28*01, and the CDR-H1, H2 and H3 were grafted onto framework sequences of the IGHV7-4-1*02. A 3D model was then generated to determine the amino acids in the original mouse framework region that are essential for antibody binding and conformation. Based on the analysis, back mutation was made on the grafting antibody, therefore, 4 additional humanized heavy chains and 4 additional light chains were generated.

For the light chain of 81C8A1, and IGKV6-21*01 is the best fit germline, and for the heavy chain of 81C8A1, IGHV2-70*04 was chosen as the humanization backbone. Humanized 81C8A1 CDR grafting antibody was then designed where the CDR-L1, L2 and L3 were grafted onto framework sequences of the IGKV6-21*01, and the CDR-H1, H2, and H3 were grafted onto framework sequences of the IGHV2-70*04. A 3D model was then generated to determine the amino acids in the original mouse framework region that are essential for antibody binding and conformation. Based on the analysis, back mutation was made on the grafting antibody, therefore, 3 additional humanized heavy chains and 4 additional light chains were generated.

The sequences of the resulting humanized sequences are listed in Tables 5-6.

TABLE 5A
Humanization of 72D1D11

| | | SEQ ID |
|---|---|---|
| Name | Sequence | NO: |
| 72D1D11 VH | QIQLVQSGPELKKPGETVKISCKASGYTFTNLGLNWVKQAPGKGLK | 5 |
| | WMGWINTAGGQPTYAEDFKGRCAFSLETSASTAYLQINNLKNEDT | |
| | ATYFCARDDGYYYTMDYWGQGTSVTVSS | |
| 72D1D11 | QVQLVQSGSELKKPGASVKVSCKASGYTFTNLGLNWVRQAPGQGL | 159 |
| VH.VI (grafted) | EWMGWINTAGGQPTYAEDFKGRFVFSLDTSVSTAYLQISSLKAEDT | |
| | AVYYCARDDGYYYTMDYWGQGTTVTVSS | |
| 72D1D11 | QVQLVQSGSELKKPGASVKVSCKASGYTFTNLGLNWVRQAPGQGL | 160 |
| VH.V2 | EWMGWINTAGGQPTYAEDFKGRCVFSLDTSVSTAYLQISSLKAEDT | |
| | AVYYCARDDGYYYTMDYWGQGTTVTVSS | |
| 72D1D11 | QVQLVQSGSELKKPGASVKVSCKASGYTFTNLGLNWVRQAPGQGL | 161 |
| VH.V3 | KWMGWINTAGGQPTYAEDFKGRCVFSLDTSVSTAYLQISSLKAEDT | |
| | AVYFCARDDGYYYTMDYWGQGTTVTVSS | |
| 72D1D11 | QIQLVQSGSELKKPGASVKVSCKASGYTFTNLGLNWVKQAPGQGLK | 162 |
| VH.V4 | WMGWINTAGGQPTYAEDFKGRCVFSLDTSVSTAYLQISSLKAEDTA | |
| | VYFCARDDGYYYTMDYWGQGTTVTVSS | |
| 72D1D11 VL | DIVMTQTPLSLPVSLGDQASISCRSSQSLVHSNGKTYLHWYLQKPG | 6 |
| | QSPKLLIYKVSNRFSGVPDRFSGSGSGTDFTLKISRVEAEDLGVYFCS | |
| | QSTHVPWTFGGGTKLEIK | |
| 72D1D11 | DIVMTQTPLSLPVTPGEPASISCRSSQSLVHSNGKTYLHWYLQKPGQ | 163 |
| VL.V1 (grafted) | SPQLLIYKVSNRFSGVPDRFSGSGSGTDFTLKISRVEAEDVGVYYCS | |
| | QSTHVPWTFGGGTKLEIK | |
| 72D1D11 | DIVMTQTPLSLPVTPGEPASISCRSSQSLVHSNGKTYLHWYLQKPGQ | 164 |
| VL.V2 | SPQLLIYKVSNRFSGVPDRFSGSGSGTDFTLKISRVEAEDVGVYFCS | |
| | QSTHVPWTFGGGTKLEIK | |
| 72D1D11 | DIVMTQSPLSLPVTPGEPASISCRSSQSLVHSNGKTYLHWYLQKPGQ | 165 |
| VL.V3 (grafted) | SPQLLIYKVSNRFSGVPDRFSGSGSGTDFTLKISRVEAEDVGVYYCS | |
| | QSTHVPWTFGGGTKLEIK | |
| 72D1D11 | DIVMTQSPLSLPVTPGEPASISCRSSQSLVHSNGKTYLHWYLQKPGQ | 166 |
| VL.V4 | SPQLLIYKVSNRFSGVPDRFSGSGSGTDFTLKISRVEAEDVGVYFCS | |
| | QSTHVPWTFGGGTKLEIK | |

TABLE 5B
Humanized antibodies from 72D1D11

| | VL | | | |
|---|---|---|---|---|
| | 72D1D11 | 72D1D11 | 72D1D11 | 72D1D11 |
| VH | VL.V1 | VL.V2 | VL.V3 | VL.V4 |
| 72D1D11 | 72D1D11-z1 | 72D1D11-z2 | 72D1D11-z3 | 72D1D11-z4 |
| VH.V1 | | | | |
| 72D1D11 | 72D1D11-z5 | 72D1D11-z6 | 72D1D11-z7 | 72D1D11-z8 |
| VH.V2 | | | | |
| 72D1D11 | 72D1D11-z9 | 72D1D11- | 72D1D11- | 72D1D11- |
| VH.V3 | | z10 | z11 | z12 |
| 72D1D11 | 72D1D11- | 72D1D11- | 72D1D11- | 72D1D11- |
| VH.V4 | z13 | z14 | z15 | z16 |

TABLE 6A
Humanization of 81C8A1

| | | SEQ ID |
|---|---|---|
| Name | Sequence | NO: |
| 81C8A1 VH | QVTLKESGPGILQPSQTLSLTCSFSGFSLSTSGMGVSWIRQPPGKGLE | 7 |
| | WLAHIYWDDDRRYNPSLKSRLTISRDTSRNQVFLQITTVDTADTATY | |
| | YCARSPYGSEAYWGQGTLVIVSA | |
| 81C8A1 VH.V1 | QVTLKESGPALVKPTQTLTLTCTFSGFSLSTSGMGVSWIRQPPGKALE | 167 |
| (grafted) | WLAHIYWDDDRRYNPSLKSRLTISKDTSKNQVVLTMTNMDPVDTA | |
| | TYYCARSPYGSEAYWGQGTLVTVSS | |
| 81C8A1 VH.V2 | QVTLKESGPALVKPTQTLTLTCTFSGFSLSTSGMGVSWIRQPPGKALE | 168 |
| | WLAHIYWDDDRRYNPSLKSRLTISRDTSKNQVVLTMTNMDPVDTA | |
| | TYYCARSPYGSEAYWGQGTLVTVSS | |
| 81C8A1 VH.V3 | QVTLKESGPALVKPTQTLTLTCTFSGFSLSTSGMGVSWIRQPPGKGL | 169 |
| | EWLAHIYWDDDRRYNPSLKSRLTISRDTSKNQVVLTITNMDPVDTA | |
| | TYYCARSPYGSEAYWGQGTLVTVSS | |
| 81C8A1 VL | MQIVLSQSPTILSAFPGEKVTMTCRASSSVSFIQWYQQKSGSSPKPWI | 8 |
| | YSPSNLASGVPTRFSGSGSGTSYSLTIGRVEAEDAATYYCQQWSSYP | |
| | WTFGGGTKVEIK | |
| 81C8A1 VL.VI | EIVLTQSPDFQSVTPKEKVTITCRASSSVSFIQWYQQKPDQSPKLWIK | 170 |
| | SPSNLASGVPSRFSGSGSGTDFTLTINSLEAEDAATYYCQQWSSYPW | |
| | TFGQGTKVEIK | |
| 81C8A1 VL.V2 | EIVLTQSPDFQSVTPKEKVTITCRASSSVSFIQWYQQKPDQSPKPWIK | 171 |
| | SPSNLASGVPSRFSGSGSGTDFTLTINSLEAEDAATYYCQQWSSYPW | |
| | TFGQGTKVEIK | |
| 81C8A1 VL.V3 | EIVLTQSPDFQSVTPKEKVTITCRASSSVSFIQWYQQKPDQSPKPWIY | 172 |
| | SPSNLASGVPSRFSGSGSGTDFTLTINSLEAEDAATYYCQQWSSYPW | |
| | TFGQGTKVEIK | |
| 81C8A1 VL.V4 | EIVLSQSPDFQSVTPKEKVTITCRASSSVSFIQWYQQKPDQSPKPWIYS | 173 |
| | PSNLASGVPSRFSGSGSGTDYTLTINSLEAEDAATYYCQQWSSYPWT | |
| | FGQGTKVEIK | |

TABLE 6B
Humanized antibodies from 81C8A1

| | VL | | | |
|---|---|---|---|---|
| | 81C8A1 | 81C8A1 | 81C8A1 | 81C8A1 |
| VH | VL.V1 | VL.V2 | VL.V3 | VL.V4 |
| 81C8A1 | 81C8A1-z1 | 81C8A1-z2 | 81C8A1-z3 | 81C8A1-z4 |
| VH.V1 | | | | |
| 81C8A1 | 81C8A1-z5 | 81C8A1-z6 | 81C8A1-z7 | 81C8A1-z8 |
| VH.V2 | | | | |
| 81C8A1 | 81C8A1-z9 | 81C8A1-z10 | 81C8A1-z11 | 81C8A1-z12 |
| VH.V3 | | | | |

### Example 7. Binding Activity of the Humanized Antibodies

Flow cytometry was conducted to confirm the binding activity of the humanized antibodies on A375 cells.

Briefly, a total number of 4×10⁴ A375 cells was first incubated with 3-fold serially diluted antibodies starting from 30 nM at 4° C. for 30 mins in FACS buffer. After washing by FACS buffer, PE conjugated-anti-human IgG antibody (eBioscience™, Invitrogen) was added to each well and incubated at 4° C. for 30 mins. Samples were washed twice with FACS buffer. The geometric mean florescence intensity (MFI) of PE was evaluated by MACSQuant Analyzer 16.

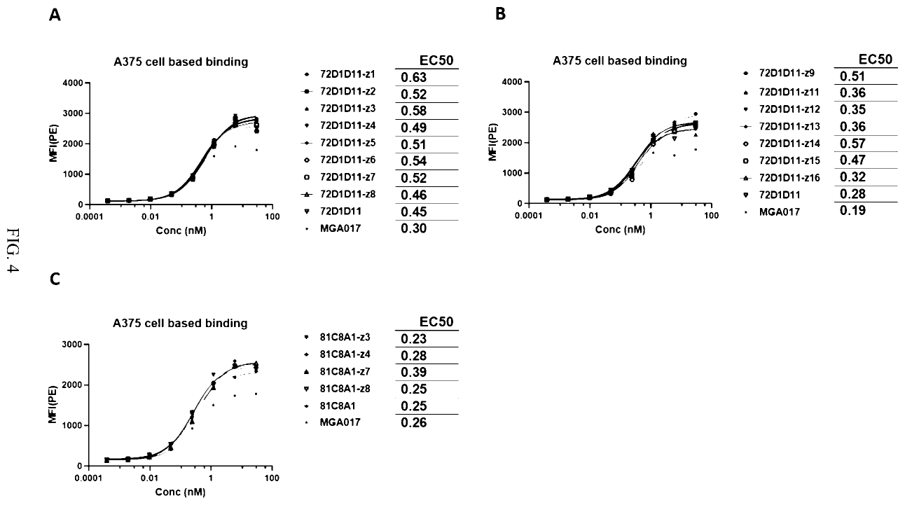

As shown in FIG. 4, all humanized 72D1D11 antibodies were comparable to the chimeric antibody on human B7-H3 binding. For the humanized 81C8A1 antibodies, 81C8A1-z3, 81C8A1-z4, 81C8A1-z7, 81C8A1-z8 showed comparable binding to the chimeric antibody.

### Example 8. PTM Removal of Chimeric 72D1D11

It has been observed that a NG residue exists on 72D1D11 VL CDR1 and a DG residue exists on 72D1D11 VH CDR3 (Kabat numbering), which are at risk of post-translational modifications (PTM) and pose challenges for future manufacturing. Therefore, this example mutated NG to QG or NA on the VL CDR1 and DG to EG on the VH CDR3 to prevent PTM. The sequences of the potential PTM removal site are listed in Table 7.

TABLE 7
PTM removal of 72D1D11

| | VL | | |
|---|---|---|---|
| | 72D1D11 | 72D1D11 | 72D1D11 |
| VH | VL | VL NG-NA | VL NG-QG |
| 72D1D11 VH | 72D1D11 | 72D1D11-p1 | 72D1D11-p2 |
| 72D1D11 VH DG-EG | 72D1D11-p3 | | |

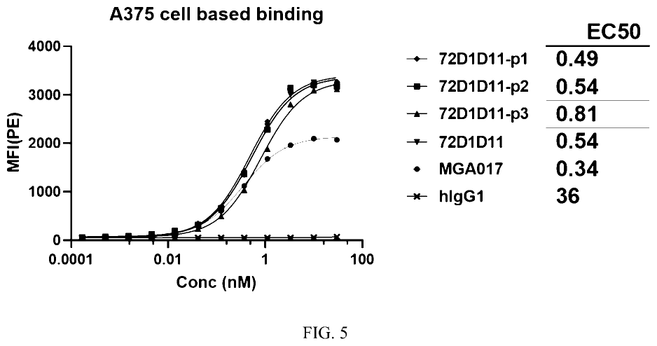

As showed in FIG. 5, 72D1D11-p1/p2 showed comparable human B7-H3 binding to the corresponding parental antibodies on A375 cells. 72D1D1I-p3 showed slight loss of human B7-H3 binding on A375 cells.

### Example 9. PTM Removal Confirm of Humanized 72D1D11

This example therefore confirmed the sequences of humanized 72D1D11 (72D1D11-z3) with removal of potential PTM site (VL CDR1 NG mutated to NA or QG; VH CDR3 DG mutated to RG or LG or AG) and sequences are listed in Table 8.

TABLE 8A
72D1D11 Humanized antibodies with
removal of potential PTM site

VL

| | 72D1D11 | 72D1D11 |
|---|---|---|
| VH | VL.V3 NG-NA | VL.V3 NG-QG |
| 72D1D11 VH.V1 | 72D1D11-z3p1 | 72D1D11-z3p2 |
| 72D1D11 VH.V1 DG-RG | 72D1D11-z3p6 | |
| 72D1D11 VH.V1 DG-LG | 72D1D11-z3p7 | |
| 72D1D11 VH.V1 DG-AG | 72D1D11-z3p8 | |

TABLE 9
Humanization of 72D1D11 with removal of potential PTM site.

| Name | Sequence | SEQ ID NO: |
|---|---|---|
| 72DID11 | QVQLVQSGSELKKPGASVKVSCKASGYTFTNLGLNWVRQA | 159 |
| VH.V1 (grafted) | PGQGLEWMGWINTAGGQPTYAEDFKGRFVFSLDTSVSTAY | |
| | LQISSLKAEDTAVYYCARDDGYYYTMDYWGQGTTVTVSS | |
| 72D1D11 | QVQLVQSGSELKKPGASVKVSCKASGYTFTNLGLNWVRQA | 174 |
| VH.V1 DG-EG | PGQGLEWMGWINTAGGQPTYAEDFKGRFVFSLDTSVSTAY | |
| | LQISSLKAEDTAVYYCARDEGYYYTMDYWGQGTTVTVSS | |
| 72D1D11 | QVQLVQSGSELKKPGASVKVSCKASGYTFTNLGLNWVRQA | 175 |
| VH.V1 DG-RG | PGQGLEWMGWINTAGGQPTYAEDFKGRFVFSLDTSVSTAY | |
| | LQISSLKAEDTAVYYCARDRGYYYTMDYWGQGTTVTVSS | |
| 72D1D11 | QVQLVQSGSELKKPGASVKVSCKASGYTFTNLGLNWVRQA | 176 |
| VH.V1 DG-LG | PGQGLEWMGWINTAGGQPTYAEDFKGRFVFSLDTSVSTAY | |
| | LQISSLKAEDTAVYYCARDLGYYYTMDYWGQGTTVTVSS | |
| 72D1D11 | QVQLVQSGSELKKPGASVKVSCKASGYTFTNLGLNWVRQA | 177 |
| VH.V1 DG-AG | PGQGLEWMGWINTAGGQPTYAEDFKGRFVFSLDTSVSTAY | |
| | LQISSLKAEDTAVYYCARDAGYYYTMDYWGQGTTVTVSS | |
| 72D1D11 | DIVMTQSPLSLPVTPGEPASISCRSSQSLVHSNGKTYLHWYL | 165 |
| VL.V3 (grafted) | QKPGQSPQLLIYKVSNRFSGVPDRFSGSGSGTDFTLKISRVEA | |
| | EDVGVYYCSQSTHVPWTFGGGTKLEIK | |
| 72D1D11 | DIVMTQSPLSLPVTPGEPASISCRSSQSLVHSNAKTYLHWYL | 178 |
| VL.V3-NG-NA | QKPGQSPQLLIYKVSNRFSGVPDRFSGSGSGTDFTLKISRVEA | |
| | EDVGVYYCSQSTHVPWTFGGGTKLEIK | |
| 72D1D11 | DIVMTQSPLSLPVTPGEPASISCRSSQSLVHSQGKTYLHWYL | 179 |
| VL.V3-NG-QG | QKPGQSPQLLIYKVSNRFSGVPDRFSGSGSGTDFTLKISRVEA | |
| | EDVGVYYCSQSTHVPWTFGGGTKLEIK | |

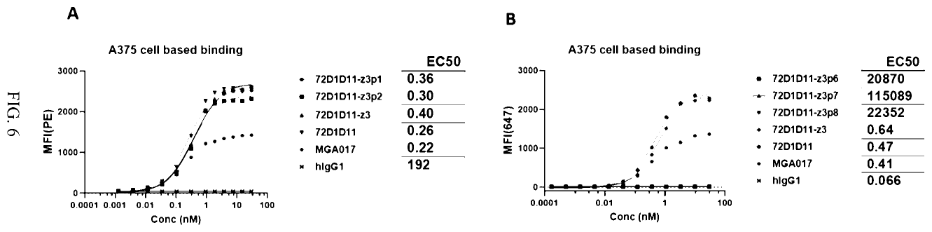

As showed in FIG. 6A, 72D1D11-z3p1 showed comparable human B7-H3 binding to the parental antibody 72D1D11-z3 on A375 cells. 72D1D11-z3p2 showed slight loss of human B7-H3 binding on A375 cells. As showed in FIG. 6B, 72D1D11-z3p6, 72D1D11-z3p7, 72D1D11-z3p8 lost the human B7-H3 binding on A375 cells.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or antigen-binding fragment thereof which has specificity to the human B7-H3 (CD276) protein and comprises a heavy chain variable region (VH) comprising a VH CDR1, a VH CDR2 and a VH CDR3, and a light chain variable region (VL) comprising a VL CDR1, a VL CDR2, and a VL CDR3, wherein:
(a) the VH CDR1 comprises the amino acid sequence of SEQ ID NO: 39;
the VH CDR2 comprises the amino acid sequence of SEQ ID NO: 40;
the VH CDR3 comprises the amino acid sequence of SEQ ID NO: 41, 42, 43, 44, 45 or 46;
the VL CDR1 comprises the amino acid sequence of SEQ ID NO: 47, 48 or 49;
the VL CDR2 comprises the amino acid sequence of SEQ ID NO: 50; and
the VL CDR3 comprises the amino acid sequence of SEQ ID NO: 51,
(b) the VH CDR1 comprises the amino acid sequence of SEQ ID NO: 52;
the VH CDR2 comprises the amino acid sequence of SEQ ID NO: 53;
the VH CDR3 comprises the amino acid sequence of SEQ ID NO: 54;
the VL CDR1 comprises the amino acid sequence of SEQ ID NO: 55;
the VL CDR2 comprises the amino acid sequence of SEQ ID NO: 56; and
the VL CDR3 comprises the amino acid sequence of SEQ ID NO: 57,
(c) the VH CDR1 comprises the amino acid sequence of SEQ ID NO: 33;
the VH CDR2 comprises the amino acid sequence of SEQ ID NO: 34;
the VH CDR3 comprises the amino acid sequence of SEQ ID NO: 35;
the VL CDR1 comprises the amino acid sequence of SEQ ID NO: 36;
the VL CDR2 comprises the amino acid sequence of SEQ ID NO: 37; and
the VL CDR3 comprises the amino acid sequence of SEQ ID NO: 38,
(d) the VH CDR1 comprises the amino acid sequence of SEQ ID NO: 25;
the VH CDR2 comprises the amino acid sequence of SEQ ID NO: 26, 27, or 28;
the VH CDR3 comprises the amino acid sequence of SEQ ID NO: 29;
the VL CDR1 comprises the amino acid sequence of SEQ ID NO: 30;
the VL CDR2 comprises the amino acid sequence of SEQ ID NO: 31; and
the VL CDR3 comprises the amino acid sequence of SEQ ID NO: 32,
(e) the VH CDR1 comprises the amino acid sequence of SEQ ID NO: 58;
the VH CDR2 comprises the amino acid sequence of SEQ ID NO: 59;
the VH CDR3 comprises the amino acid sequence of SEQ ID NO: 60;
the VL CDR1 comprises the amino acid sequence of SEQ ID NO: 61;
the VL CDR2 comprises the amino acid sequence of SEQ ID NO: 62; and
the VL CDR3 comprises the amino acid sequence of SEQ ID NO: 63,
(f) the VH CDR1 comprises the amino acid sequence of SEQ ID NO: 64;
the VH CDR2 comprises the amino acid sequence of SEQ ID NO: 65 or 66;
the VH CDR3 comprises the amino acid sequence of SEQ ID NO: 67;
the VL CDR1 comprises the amino acid sequence of SEQ ID NO: 68;
the VL CDR2 comprises the amino acid sequence of SEQ ID NO: 69; and
the VL CDR3 comprises the amino acid sequence of SEQ ID NO: 70,
(g) the VH CDR1 comprises the amino acid sequence of SEQ ID NO: 71;
the VH CDR2 comprises the amino acid sequence of SEQ ID NO: 72, 73 or 74;
the VH CDR3 comprises the amino acid sequence of SEQ ID NO: 75;
the VL CDR1 comprises the amino acid sequence of SEQ ID NO: 76;
the VL CDR2 comprises the amino acid sequence of SEQ ID NO: 77; and
the VL CDR3 comprises the amino acid sequence of SEQ ID NO: 78,
(h) the VH CDR1 comprises the amino acid sequence of SEQ ID NO: 79;
the VH CDR2 comprises the amino acid sequence of SEQ ID NO: 80;
the VH CDR3 comprises the amino acid sequence of SEQ ID NO: 81;
the VL CDR1 comprises the amino acid sequence of SEQ ID NO: 82;
the VL CDR2 comprises the amino acid sequence of SEQ ID NO: 83; and
the VL CDR3 comprises the amino acid sequence of SEQ ID NO: 84,
(i) the VH CDR1 comprises the amino acid sequence of SEQ ID NO: 85;
the VH CDR2 comprises the amino acid sequence of SEQ ID NO: 86 or 87;
the VH CDR3 comprises the amino acid sequence of SEQ ID NO: 88;
the VL CDR1 comprises the amino acid sequence of SEQ ID NO: 89;
the VL CDR2 comprises the amino acid sequence of SEQ ID NO: 90; and
the VL CDR3 comprises the amino acid sequence of SEQ ID NO: 91,
(j) the VH CDR1 comprises the amino acid sequence of SEQ ID NO: 92;
the VH CDR2 comprises the amino acid sequence of SEQ ID NO: 93, 94, 95 or 96;
the VH CDR3 comprises the amino acid sequence of SEQ ID NO: 97;
the VL CDR1 comprises the amino acid sequence of SEQ ID NO: 98;
the VL CDR2 comprises the amino acid sequence of SEQ ID NO: 99 or 100; and
the VL CDR3 comprises the amino acid sequence of SEQ ID NO: 101,
(k) the VH CDR1 comprises the amino acid sequence of SEQ ID NO: 102;
the VH CDR2 comprises the amino acid sequence of SEQ ID NO: 103;
the VH CDR3 comprises the amino acid sequence of SEQ ID NO: 104;
the VL CDR1 comprises the amino acid sequence of SEQ ID NO: 105;
the VL CDR2 comprises the amino acid sequence of SEQ ID NO: 106; and
the VL CDR3 comprises the amino acid sequence of SEQ ID NO: 107,
(l) the VH CDR1 comprises the amino acid sequence of SEQ ID NO: 108;
the VH CDR2 comprises the amino acid sequence of SEQ ID NO: 109;
the VH CDR3 comprises the amino acid sequence of SEQ ID NO: 110, 111 or 112;
the VL CDR1 comprises the amino acid sequence of SEQ ID NO: 113, 114 or 115;
the VL CDR2 comprises the amino acid sequence of SEQ ID NO: 116; and
the VL CDR3 comprises the amino acid sequence of SEQ ID NO: 117, or
(m) the VH CDR1 comprises the amino acid sequence of SEQ ID NO: 182;
the VH CDR2 comprises the amino acid sequence of SEQ ID NO: 183, 184, 185, or 186;
the VH CDR3 comprises the amino acid sequence of SEQ ID NO: 187;
the VL CDR1 comprises the amino acid sequence of SEQ ID NO: 188, 189, 190, or 191;
the VL CDR2 comprises the amino acid sequence of SEQ ID NO: 192; and
the VL CDR3 comprises the amino acid sequence of SEQ ID NO: 193.

2. The antibody or antigen-binding fragment thereof of claim 1, wherein:
(c) the VH CDR1 comprises the amino acid sequence of SEQ ID NO: 39;
the VH CDR2 comprises the amino acid sequence of SEQ ID NO: 40;
the VH CDR3 comprises the amino acid sequence of SEQ ID NO: 41, 42, 43, 44, 45 or 46;
the VL CDR1 comprises the amino acid sequence of SEQ ID NO: 47, 48 or 49;
the VL CDR2 comprises the amino acid sequence of SEQ ID NO: 50; and
the VL CDR3 comprises the amino acid sequence of SEQ ID NO: 51.

3. The antibody or antigen-binding fragment thereof of claim 2, wherein:
the VH CDR1 comprises the amino acid sequence of SEQ ID NO: 39;
the VH CDR2 comprises the amino acid sequence of SEQ ID NO: 40;
the VH CDR3 comprises the amino acid sequence of SEQ ID NO: 41;
the VL CDR1 comprises the amino acid sequence of SEQ ID NO: 47;
the VL CDR2 comprises the amino acid sequence of SEQ ID NO: 50; and
the VL CDR3 comprises the amino acid sequence of SEQ ID NO: 51.

4. The antibody or antigen-binding fragment thereof of claim 3, wherein the VH comprises an amino acid sequence selected from the group consisting of SEQ ID NO: 5 and 159-162 and the VL comprises an amino acid sequence selected from the group consisting of SEQ ID NO: 6 and 163-166.

5. The antibody or antigen-binding fragment thereof of claim 3, wherein the VH comprises the amino acid sequence of SEQ ID NO: 159 and the VL comprises the amino acid sequence of SEQ ID NO: 165.

6. The antibody or antigen-binding fragment thereof of claim 2, wherein:
the VH CDR1 comprises the amino acid sequence of SEQ ID NO: 39;
the VH CDR2 comprises the amino acid sequence of SEQ ID NO: 40;
the VH CDR3 comprises the amino acid sequence of SEQ ID NO: 41;
the VL CDR1 comprises the amino acid sequence of SEQ ID NO: 48 or 49;
the VL CDR2 comprises the amino acid sequence of SEQ ID NO: 50; and
the VL CDR3 comprises the amino acid sequence of SEQ ID NO: 51.

7. The antibody or antigen-binding fragment thereof of claim 6, wherein the VH comprises the amino acid of SEQ ID NO: 159 and the VL comprises the amino acid sequence of SEQ ID NO: 178.

8. The antibody or antigen-binding fragment thereof of claim 6, wherein the VH comprises the amino acid of SEQ ID NO: 159 and the VL comprises the amino acid sequence of SEQ ID NO: 179.

9. The antibody or antigen-binding fragment thereof of claim 1, wherein:
the VH CDR1 comprises the amino acid sequence of SEQ ID NO: 52;
the VH CDR2 comprises the amino acid sequence of SEQ ID NO: 53;
the VH CDR3 comprises the amino acid sequence of SEQ ID NO: 54;
the VL CDR1 comprises the amino acid sequence of SEQ ID NO: 55;
the VL CDR2 comprises the amino acid sequence of SEQ ID NO: 56; and
the VL CDR3 comprises the amino acid sequence of SEQ ID NO: 57.

10. The antibody or antigen-binding fragment thereof of claim 9, wherein the VH comprises an amino acid sequence selected from the group consisting of SEQ ID NO: 7 and 167-169 and the VL comprises an amino acid sequence selected from the group consisting of SEQ ID NO: 8 and 170-173.

11. The antibody or antigen-binding fragment thereof of claim 1, wherein:
the VH CDR1 comprises the amino acid sequence of SEQ ID NO: 33;
the VH CDR2 comprises the amino acid sequence of SEQ ID NO: 34;
the VH CDR3 comprises the amino acid sequence of SEQ ID NO: 35;
the VL CDR1 comprises the amino acid sequence of SEQ ID NO: 36;
the VL CDR2 comprises the amino acid sequence of SEQ ID NO: 37; and
the VL CDR3 comprises the amino acid sequence of SEQ ID NO: 38.

12. The antibody or antigen-binding fragment thereof of claim 9, wherein the VH comprises the amino acid sequence of SEQ ID NO: 3 and the VL comprises the amino acid sequence of SEQ ID NO: 4.

13. An antibody or antigen-binding fragment thereof which has specificity to the human B7-H3 (CD276) protein and comprises a heavy chain variable region (VH) comprising a VH CDR1, a VH CDR2 and a VH CDR3, and a light chain variable region (VL) comprising a VL CDR1, a VL CDR2, and a VL CDR3, wherein:
(a) the VH CDR1 comprises the amino acid sequence of SEQ ID NO: 149;
the VH CDR2 comprises the amino acid sequence of SEQ ID NO: 150, 151 or 152;
the VH CDR3 comprises the amino acid sequence of SEQ ID NO: 153 or 154;
the VL CDR1 comprises the amino acid sequence of SEQ ID NO: 155;
the VL CDR2 comprises the amino acid sequence of SEQ ID NO: 156; and
the VL CDR3 comprises the amino acid sequence of SEQ ID NO: 157 or 158,
(b) the VH CDR1 comprises the amino acid sequence of SEQ ID NO: 126;
the VH CDR2 comprises the amino acid sequence of SEQ ID NO: 127 or 128;
the VH CDR3 comprises the amino acid sequence of SEQ ID NO: 129, 130 or 131;
the VL CDR1 comprises the amino acid sequence of SEQ ID NO: 132;
the VL CDR2 comprises the amino acid sequence of SEQ ID NO: 133; and
the VL CDR3 comprises the amino acid sequence of SEQ ID NO: 134,
(c) the VH CDR1 comprises the amino acid sequence of SEQ ID NO: 135;
the VH CDR2 comprises the amino acid sequence of SEQ ID NO: 136 or 137;
the VH CDR3 comprises the amino acid sequence of SEQ ID NO: 138;
the VL CDR1 comprises the amino acid sequence of SEQ ID NO: 139;
the VL CDR2 comprises the amino acid sequence of SEQ ID NO: 140; and
the VL CDR3 comprises the amino acid sequence of SEQ ID NO: 141, or
(d) the VH CDR1 comprises the amino acid sequence of SEQ ID NO: 142;
the VH CDR2 comprises the amino acid sequence of SEQ ID NO: 143;
the VH CDR3 comprises the amino acid sequence of SEQ ID NO: 144;
the VL CDR1 comprises the amino acid sequence of SEQ ID NO: 145;
the VL CDR2 comprises the amino acid sequence of SEQ ID NO: 146; and
the VL CDR3 comprises the amino acid sequence of SEQ ID NO: 147 or 148.

14. The antibody or antigen-binding fragment thereof of claim 13, wherein:
the VH CDR1 comprises the amino acid sequence of SEQ ID NO: 149;
the VH CDR2 comprises the amino acid sequence of SEQ ID NO: 150; 151 or 152;
the VH CDR3 comprises the amino acid sequence of SEQ ID NO: 153 or 154;
the VL CDR1 comprises the amino acid sequence of SEQ ID NO: 155;
the VL CDR2 comprises the amino acid sequence of SEQ ID NO: 156; and
the VL CDR3 comprises the amino acid sequence of SEQ ID NO: 157 or 158.

15. The antibody or antigen-binding fragment thereof of claim 14, wherein:
the VH CDR1 comprises the amino acid sequence of SEQ ID NO: 149;
the VH CDR2 comprises the amino acid sequence of SEQ ID NO: 150;
the VH CDR3 comprises the amino acid sequence of SEQ ID NO: 153;
the VL CDR1 comprises the amino acid sequence of SEQ ID NO: 155;
the VL CDR2 comprises the amino acid sequence of SEQ ID NO: 156; and
the VL CDR3 comprises the amino acid sequence of SEQ ID NO: 157.

16. The antibody or antigen-binding fragment thereof of claim 15, wherein the VH comprises the amino acid sequence of SEQ ID NO: 124 and the VL comprises the amino acid sequence of SEQ ID NO: 125.

17. The antibody or antigen-binding fragment thereof of claim 13, wherein:
the VH CDR1 comprises the amino acid sequence of SEQ ID NO: 126;
the VH CDR2 comprises the amino acid sequence of SEQ ID NO: 127 or 128;
the VH CDR3 comprises the amino acid sequence of SEQ ID NO: 129, 130 or 131;
the VL CDR1 comprises the amino acid sequence of SEQ ID NO: 132;
the VL CDR2 comprises the amino acid sequence of SEQ ID NO: 133; and
the VL CDR3 comprises the amino acid sequence of SEQ ID NO: 134.

18. The antibody or antigen-binding fragment thereof of claim 17, wherein the VH comprises the amino acid sequence of SEQ ID NO: 118 and the VL comprises the amino acid sequence of SEQ ID NO: 119.

19. The antibody or antigen-binding fragment thereof of claim 13, wherein:
the VH CDR1 comprises the amino acid sequence of SEQ ID NO: 135;
the VH CDR2 comprises the amino acid sequence of SEQ ID NO: 136 or 137;
the VH CDR3 comprises the amino acid sequence of SEQ ID NO: 138;
the VL CDR1 comprises the amino acid sequence of SEQ ID NO: 139;
the VL CDR2 comprises the amino acid sequence of SEQ ID NO: 140; and
the VL CDR3 comprises the amino acid sequence of SEQ ID NO: 141.

20. The antibody or antigen-binding fragment thereof of claim 19, wherein the VH comprises the amino acid sequence of SEQ ID NO: 120 and the VL comprises the amino acid sequence of SEQ ID NO: 121.

21. The antibody or antigen-binding fragment thereof of claim 13, wherein:
the VH CDR1 comprises the amino acid sequence of SEQ ID NO: 142;
the VH CDR2 comprises the amino acid sequence of SEQ ID NO: 143;
the VH CDR3 comprises the amino acid sequence of SEQ ID NO: 144,
the VL CDR1 comprises the amino acid sequence of SEQ ID NO: 145;
the VL CDR2 comprises the amino acid sequence of SEQ ID NO: 146; and
the VL CDR3 comprises the amino acid sequence of SEQ ID NO: 147 or 148.

22. The antibody or antigen-binding fragment thereof of claim 21, wherein the VH comprises the amino acid sequence of SEQ ID NO: 122 and the VL comprises the amino acid sequence of SEQ ID NO: 123.

23. The antibody or fragment thereof of any one of claims 1-22, wherein the antibody or fragment thereof is a bivalent Fab antibody, or a fragment selected from the group consisting of F(ab′)2, F(ab)2, Fab′, Fab, FV, and scFV.

24. A multispecific antibody comprising an antigen-binding fragment of claim 1 and one or more antibody or antigen-binding fragment having binding specificity to a target antigen that is not B7-H3.

25. A chimeric antigen receptor (CAR) comprising an antigen-binding fragment of claim 1, a transmembrane domain, a costimulatory domain, and a CD3ξ intracellular domain.

26. One or more polynucleotide(s) encoding the antibody or antigen-binding fragment thereof of claim 1.

27. The polynucleotide(s) of claim 26, which is one or more mRNA.

28. The polynucleotide(s) of claim 27, wherein the mRNA is chemically modified.

29. A cell comprising the polynucleotide(s) of any one of claims 26-28.

30. An antibody-drug conjugate, comprising:
(a) an antibody or fragment thereof of claim 1; and
(b) a conjugation moiety conjugated to the antibody or fragment thereof, wherein the conjugation moiety is selected from: a detectable marker, a drug, a toxin, a cytokine, a radionuclide, an enzyme, or a combination thereof.

31. (canceled)

32. A method of treating cancer in a patient in need thereof, comprising administering to the patient an effective amount of the antibody or antigen-binding fragment thereof of claim 1.

33. (canceled)
